SEQUENCE LISTING (61 sequences)

SEQ ID NO: 1 - protein, 26 aa, Homo sapiens
KTWVHYIAAEEEDWDYAPLVLAPDDR

SEQ ID NO: 2 - protein, 56 aa, Homo sapiens
ITDVRPLYSRRLPKGVKHLKDFPILPGEIFKYKWTVTVEDGPTKSDPRCLTRYYSS

SEQ ID NO: 3 - protein, 37 aa, Homo sapiens
AYWYILSIGAQTDFLSVFFSGYTFKHKMVYEDTLTLF

SEQ ID NO: 4 - protein, 60 aa, Homo sapiens
VEMKKEDFDIYDEDENQSPRSFQKKTRHYFIAAVERLWDYGMSSSPHVLRNRAQSGSVPQ

SEQ ID NO: 5 - protein, 60 aa, Homo sapiens
ISYEEDQRQGAEPRKNFVKPNETKTYFWKVQHHMAPTKDEFDCKAWAYFSDVDLEKDVHS

SEQ ID NO: 6 - protein, 40 aa, Homo sapiens
IRWYLLSMGSNENIHSIHFSGHVFTVRKKEEYKMALYNLY

SEQ ID NO: 7 - protein, 80 aa, Homo sapiens
STLRMELMGCDLNSCSMPLGMESKAISDAQITASSYFTNMFATWSPSKARLHLQGRSNAWRPQVNNPKEWLQVDFQKTMK

SEQ ID NO: 8 - protein, 50 aa, Homo sapiens
VKVFQGNQDSFTPVVNSLDPPLLTRYLRIHPQSWVHQIALRMEVLGCEAQ

SEQ ID NO: 9 - protein, 20 aa, Homo sapiens
FKKVVFQEFTDGSFTQPLYR

SEQ ID NO: 10 - protein, 20 aa, Homo sapiens
DGSFTQPLYRGELNEHLGLL

SEQ ID NO: 11 - protein, 20 aa, Homo sapiens
GELNEHLGLLGPYIRAEVED

SEQ ID NO: 12 - protein, 20 aa, Homo sapiens
GPYIRAEVEDNIMVTFRNQA

SEQ ID NO: 13 - protein, 20 aa, Homo sapiens
NIMVTFRNQASRPYSFYSSL

SEQ ID NO: 14 - protein, 20 aa, Homo sapiens
SRPYSFYSSLISYEEDQRQG

SEQ ID NO: 15 - protein, 20 aa, Homo sapiens
ISYEEDQRQGAEPRKNFVKP

SEQ ID NO: 16 - protein, 20 aa, Homo sapiens
AEPRKNFVKPNETKTYFWKV

SEQ ID NO: 17 - protein, 20 aa, Homo sapiens
NETKTYFWKVQHHMAPTKDE

SEQ ID NO: 18 - protein, 20 aa, Homo sapiens
QHHMAPTKDEFDCKAWAYFS

SEQ ID NO: 19 - protein, 20 aa, Homo sapiens
FDCKAWAYFSDVDLEKDVHS

SEQ ID NO: 20 - protein, 20 aa, Homo sapiens
DVDLEKDVHSGLIGPLLVCH

SEQ ID NO: 21 - protein, 20 aa, Homo sapiens
GLIGPLLVCHTNTLNPAHGR

SEQ ID NO: 22 - protein, 20 aa, Homo sapiens
TNTLNPAHGRQVTVQEFALF

SEQ ID NO: 23 - protein, 20 aa, Homo sapiens
QVTVQEFALFFTIFDETKSW

SEQ ID NO: 24 - protein, 20 aa, Homo sapiens
FTIFDETKSWYFTENMERNC

SEQ ID NO: 25 - protein, 20 aa, Homo sapiens
YFTENMERNCRAPCNIQMED

SEQ ID NO: 26 - protein, 20 aa, Homo sapiens
RAPCNIQMEDPTFKENYRFH

SEQ ID NO: 27 - protein, 20 aa, Homo sapiens
PTFKENYRFHAINGYIMDTL

SEQ ID NO: 28 - protein, 20 aa, Homo sapiens
AINGYIMDTLPGLVMAQDQR

SEQ ID NO: 29 - protein, 20 aa, Homo sapiens
PGLVMAQDQRIRWYLLSMGS

SEQ ID NO: 30 - protein, 20 aa, Homo sapiens
IRWYLLSMGSNENIHSIHFS

SEQ ID NO: 31 - protein, 20 aa, Homo sapiens
NENIHSIHFSGHVFTVRKKE

SEQ ID NO: 32 - protein, 20 aa, Homo sapiens
GHVFTVRKKEEYKMALYNLY

SEQ ID NO: 33 - protein, 20 aa, Homo sapiens
EYKMALYNLYPGVFETVEML

SEQ ID NO: 34 - protein, 20 aa, Homo sapiens
PGVFETVEMLPSKAGIWRVE

SEQ ID NO: 35 - protein, 20 aa, Homo sapiens
PSKAGIWRVECLIGEHLHAG

SEQ ID NO: 36 - protein, 20 aa, Homo sapiens
CLIGEHLHAGMSTLFLVYSN

SEQ ID NO: 37 - protein, 20 aa, Homo sapiens
DLNSCSMPLGMESKAISDAQ

SEQ ID NO: 38 - protein, 20 aa, Homo sapiens
MESKAISDAQITASSYFTNM

SEQ ID NO: 39 - protein, 20 aa, Homo sapiens
ITASSYFTNMFATWSPSKAR

SEQ ID NO: 40 - protein, 20 aa, Homo sapiens
FATWSPSKARLHLQGRSNAW

SEQ ID NO: 41 - protein, 20 aa, Homo sapiens
LHLQGRSNAWRPQVNNPKEW

SEQ ID NO: 42 - protein, 20 aa, Homo sapiens
RPQVNNPKEWLQVDFQKTMK

SEQ ID NO: 43 - protein, 20 aa, Homo sapiens
LQVDFQKTMKVTGVTTQGVK

SEQ ID NO: 44 - protein, 20 aa, Homo sapiens
VTGVTTQGVKSLLTSMYVKE

SEQ ID NO: 45 - protein, 20 aa, Homo sapiens
SLLTSMYVKEFLISSSQDGH

SEQ ID NO: 46 - protein, 20 aa, Homo sapiens
FLISSSQDGHQWTLFFQNGK

SEQ ID NO: 47 - protein, 20 aa, Homo sapiens
QWTLFFQNGKVKVFQGNQDS

SEQ ID NO: 48 - protein, 20 aa, Homo sapiens
VKVFQGNQDSFTPVVNSLDP

SEQ ID NO: 49 - protein, 20 aa, Homo sapiens
FTPVVNSLDPPLLTRYLRIH

SEQ ID NO: 50 - protein, 20 aa, Homo sapiens
PLLTRYLRIHPQSWVHQIAL

SEQ ID NO: 51 - protein, 20 aa, Homo sapiens
PQSWVHQIALRMEVLGCEAQ

SEQ ID NO: 52 - protein, 13 aa, Homo sapiens
RMEVLGCEAQDLY

SEQ ID NO: 53 - protein, 20 aa, Homo sapiens
ITRTTLQSDQEEIDYDDTIS

SEQ ID NO: 54 - protein, 20 aa, Homo sapiens
EEIDYDDTISVEMKKEDFDI

SEQ ID NO: 55 - protein, 20 aa, Homo sapiens
VEMKKEDFDIYDEDENQSPR

SEQ ID NO: 56 - protein, 20 aa, Homo sapiens
YDEDENQSPRSFQKKTRHYF

SEQ ID NO: 57 - protein, 20 aa, Homo sapiens
SFQKKTRHYFIAAVERLWDY

SEQ ID NO: 58 - protein, 20 aa, Homo sapiens
IAAVERLWDYGMSSSPHVLR

SEQ ID NO: 59 - protein, 20 aa, Homo sapiens
GMSSSPHVLRNRAQSGSVPQ

SEQ ID NO: 60 - protein, 20 aa, Homo sapiens
NRAQSGSVPQFKKVVFQEFT

SEQ ID NO: 61 - protein, 20 aa, Homo sapiens
STLRMELMGCDLNSCSMPLG

(19) United States
(12) Patent Application Publication
(10) Pub. No.: US 2004/0096456 A1
(43) Pub. Date: May 20, 2004

(54) Method to treat hemophilia

(76) Inventor: Bianca M Conti-fine, Minneapolis, MN (US)

Correspondence Address: Schwegman Lundberg Woessner & Kluth, P O Box 2938, Minnapolis, MN, 55402, US

(21) Appl. No.: 10/433,273

(22) PCT Filed: Nov. 30, 2001
(86) PCT No.: PCT/US01/44945

(51) Int. Cl. A61K 39/00; C07K 14/47

(52) U.S. Cl. 424/185.1; 530/324; 530/326

(57) ABSTRACT

Isolated and purified peptides and variants thereof, as well as DNA encoding those peptides, useful to prevent or treat antibody inhibitors of factor VIII, are provided.

### CROSS REFERENCE TO RELATED APPLICATIONS

This application claims the benefit of the filing date of U.S. application Serial No. 60/250,430, filed Dec. 1, 2000, under 35 U.S.C. § 119(e).

### STATEMENT OF GOVERNMENT RIGHTS

The present invention was made with the support of the United States Government (grant HL61922 from the National Heart, Lung and Blood Institute). The Government may have certain rights in the invention.

### BACKGROUND OF THE INVENTION

Ideal treatments for a pathological condition or disease caused by an undesirable immune response would specifically affect antigen-specific T and B cells. Antigen specific tolerization of T cells can be obtained by delivery of the antigen through routes, such as oral, intraperitoneal and nasal administration, that downregulate, rather than activate, CD4+ responses (Natzinger, 1994; Nossal, 1995). Tolerization of T cells by those routes has proven effective for the prevention and/or treatment of CD4+ T cell mediated autoimmune diseases, e.g., experimental autoimmune encephalomyelitis (EAE) (Metzler et al., 1993; Miller et al., 1994; Genain et al., 1996; Al-Sabbagh et al., 1996), collagen-induced arthritis (Al-Sabbagh et al., 1996), experimental uveitis (Dick et al., 1993), and myasthenia gravis (Karachunski et al., 1997). Moreover, the administration of the antigen by these methods reduced or inhibited the immune response specific for the particular antigen administered. For example, aerosol administration of myelin basic protein (MBP) to MBP-immunized rats that had developed relapsing EAE decreased the intensity of the immune response to MBP and the severity of the attacks (Al-Sabbagh et al., 1996). Spleen T cells from rats that had inhaled MBP transferred protection to naive animals (Al-Sabbagh et al., 1996).

It is unclear whether similar approaches could be used for antibody (Ab)-mediated diseases for two reasons. First, while effective at reducing antigen-specific CD4+ responses, administration of antigen through routes that downregulate CD4+ responses may directly stimulate B cells specific for the administered antigen (Kuper et al., 1992; Liu et al., 1993; Husby et al., 1994; Neutra et al., 1996). This stimulation may have disastrous consequences, as has been shown in marmoset EAE (Genain et al., 1996), where intraperitoneal administration of myelin resulted in CD4+ tolerance to myelin, but also in an acute, fatal form of EAE. The fatal form of EAE was characterized by antibody specific for the myelin oligodendrocyte glycoprotein. Second, administration of antigen through routes that stimulate Th2 cells and downregulate pro-inflammatory Th1 cells can stimulate antibody synthesis (Neutra et al., 1996; Abbas et al., 1996), and cause exacerbation rather than improvement of antibody-mediated autoimmune diseases.

Hemophilia A is an X-linked bleeding disorder that affects 1 in 5,000-10,000 males (Hoyer et al., 1990). Hemophilia A patients genetically lack coagulation factor VIII (fV1) (Hoyer et al., 1994; Sadler et al., 1987; Kazazian et al., 1995). Patients with severe hemophilia A have fVIII activity which is less than 1% of normal (Naylor-et al., 1993). fVIII is a cofactor in a crucial step in hemostasis. Its absence causes severe bleedings after minimal traumas, or even spontaneously. Hemophilia A patients require regular administrations of human fVIII to treat their bleeding episodes.

Patients with severe hemophilia A are not immunologically tolerant to fVIII. When treated with fVIII products to control their bleedings, they may develop antibodies to fVIII which block its function (inhibitors) (Hoyer et al., 1995). fVIII inhibitors develop in 20-25% of patients with hemophilia A (Hoyer et al., 1995; Kreuz et al., 1996; Aledort et al., 1994; Ehrenforth et al., 1990), and they make the patients' treatment very difficult. fVIII inhibitors also develop in subjects who do not have hemophilia A, in a disorder known as acquired hemophilia. This is a rare but frequently fatal disease in which fVIII is the target of an autoimmune response (Bouvry et al., 1994).

The cost of caring for hemophiliacs with inhibitors is extraordinarily high (typically, $100,000-$250,000 per patient/year) (Aledort et al, 1996). Because they cannot rely on standard fVIII replacement therapy, they must either undergo the lengthy and extremely costly procedure of high-dose immune tolerance induction, or they must rely on “bypass” therapy to circumvent pre-formed inhibitors. The efficacy of the latter in controlling hemorrhages is often uncertain. Immune tolerance induction by daily administration of high doses of fVIII, over many months, costs up to $1,000,000/patient and its success rate is only 70% or less (Aledort et al., 1994; Nlsson et al., 1998; Mariani et al., 1995).

The availability of fVIII replacement therapy has greatly increased the hemophilia A patients' life expectancy, which now approaches that of normal persons (Triemstra et al., 1995). Unfortunately, even the most modern replacement therapy has not reduced the risk of developing inhibitors. Studies on the use of recombinant fVIII have shown that the incidence of inhibitor formation in infants and children with severe hemophilia A was even higher than previously thought (29%) (Lee, 1999). Thus, it can be expected that even in the up-coming era of gene therapy for hemophilia, inhibitors will continue to be a significant issue limiting effective treatment for many patients.

Thus, although hemophilia A is a rare disease, its financial and human costs make it far more important than it might be judged if only the number of affected patients were considered. The limitations to effective management of hemophilia patients caused by inhibitors support the continuing need for efficacious, safe, convenient, and cost-effective means of immune tolerance induction, e.g., methods which could specifically prevent the development of inhibitors before the first exposure to fVIII in infancy or specifically reduce the ongoing synthesis of antibody inhibitors would represent a significant therapeutic advance.

### SUMMARY OF THE INVENTION

The present invention provides a therapeutic method comprising the administration of at least one epitope peptide comprising a universal and/or immunodominant epitope sequence from a portion (fragment) of factor VIII (fVIII) to a mammal in need of such treatment, e.g., a mammal at risk of developing antibody inhibitors to fVIII, a biologically active fragment thereof or a functional equivalent thereof, or having antibody inhibitors to fVIII, a biologically active fragment thereof or a functional equivalent thereof, e.g., a mammal with hemophilia A or acquired hemophilia. The method is effective to specifically tolerize, enhance the activity or levels of modulatory (regulatory) T (CD4+) cells that inhibit or down regulate the immune response to factor VIII, i.e, the synthesis of antibodies specific for fVIII, down regulate the priming and/or activity of, fVIII antigen-specific T cells, and/or alter aberrant (pathogenic) antibody production in the mammal. The pathogenic antibodies, i.e., “fVIII inhibitors”, are those which are specific for the fVIII, a biologically active fragment thereof or a functional equivalent thereof, used to treat bleeding in hemophilia A patients. Thus, the fVIII which is administered may be native or recombinant protein or in a DNA vector that encodes fVIII, biologically active fragment or a functional equivalent thereof, and/or is synthesized by the host as a result of gene therapy. As used herein, a “biologically active fragment a functional equivalent” of fVIII is a molecule which has the procoagulant activity of fVIII and includes forms of fVIII which do not comprise the B domain (see FIG. 1) or altered forms of fVIII which are less immunogenic.

Antibody synthesis is controlled by T cells and in mammals there are limited sets of epitopes for each antigen that dominate the T cell response, referred to as immunodominant T cell epitope sequences (hereinafter “immunodominant epitope sequences”). Moreover, in humans, CD4+ cells recognize universal, immunodominant epitope sequences. As T cell epitopes may comprise as few as 7 amino acid residues corresponding to an amino acid sequence present in a particular antigen, peptides having at least about 7 amino acid residues may be useful to tolerize, or down regulate the priming and/or activity of, T cells (e.g., CD4+ cells) specific for the peptide and its corresponding antigen. Thus, immunodominant and/or universal epitope peptides may be administered so as to regulate a mammal's T cell and thus aberrant antibody response.

As described hereinbelow, a mouse model of hemophilia A was employed to demonstrate that fVIII-specific peptide based tolerance could be achieved. The peptides that were administered were 20 residue synthetic sequences of human fVIII that form epitopes for the mouse CD4⁺ cells which effectively protected the mice from development of anti-fVIII antibodies after administration of fVIII intravenously at doses comparable to those used for treatment of hemophilia A patients. Moreover, the sequence regions of the A3 and C2 domains of human fVIII were identified which are recognized by hemophilia patients with or without inhibitors, and by healthy subjects. Some sequence regions were strongly recognized by all inhibitor patients, whereas they were recognized inconsistently by patients without inhibitors and by healthy controls. Other sequence regions were recognized by most patients, irrespective of their inhibitor status, and by most controls. Further, based on the structural similarity between the A2 and A3 domains, and the sequence location of the universal epitopes and their relationship to the sequence regions forming binding sites for antibody inhibitors, regions of the A2 domain that likely form universal CD4⁺ epitopes were identified. These sequences are immunodominant, universal epitopes for CD4+ T cells and are ideally suited for induction of immune tolerance to fVIII to prevent or inhibit the production of inhibitors in hemophilia A, e.g., by inducing immune tolerance by acting on fVIII-specific CD4+ T cells. In particular, a pool of those sequences (synthetic, biosynthetic, or directly synthesized by the patient as a result of gene transfer) may be employed to induce tolerance to fVIII in hemophilia A patients and in patients with acquired hemophilia.

The demonstration in humans of universal, immunodominant CD4⁺ epitopes is important for the development of immune tolerance procedures for the prevention and treatment of fVIII inhibitors. Identification and synthesis of universal CD4⁺ epitope sequences of fVIII would allow their use for tolerization procedures suitable for the treatment of any patients. Universal CD4⁺ epitope sequences would be suitable also for preventing appearance of inhibitors as the epitope repertoire recognized by fVIII sensitized CD4⁺ cells in each patient would not need to be identified. Administration of these peptides could tolerize the CD4⁺ clones potentially reactive to fVIII sequences prior to the first therapeutic exposure to fVIII.

Although the invention is not limited to a particular route of peptide administration, subcutaneous, intravenous and respiratory, e.g., nasal (upper) or lower respiratory tract, administration are promising tolerizing routes when using an epitope peptide, since the peptide does not need to overcome the proteolytic barriers present in the digestive system, and crosses the epithelia more readily than larger polypeptide molecules. Thus, synthetic CD4+ epitope sequences may be more effective than the whole or native antigen for tolerance induction. Moreover, the peptides of the invention can be prepared in large quantities and in high purity by chemical syntheses and thus are much less expensive and more readily obtained than a preparation comprising isolated autoantigen. Further, the delivery of epitope peptides to other mucosal surfaces, e.g., in the intestine, the mouth, the genital tract, and the eye, may also be employed in the practice of the methods of the invention, although the invention is not limited to administration by mucosal routes.

The administration of peptides to mucosal surfaces or systemically can result in a state of peripheral tolerance, a situation characterized by the fact that immune responses in non-mucosal tissues do not develop even if the peptide initially contacted with the mucosa is reintroduced, or its corresponding antigen is introduced or interacts with the immune system in the organism by a nonmucosal route. Since this phenomenon is exquisitely specific for the peptide, and thus does not influence the development of systemic immune responses against other antigens, its use is particular envisioned for preventing and treating illnesses associated or resulting from the development of exaggerated immunological reactions against specific antigens encountered in nonmucosal tissues. For example, one embodiment of the invention is a method in which a mammal is contacted with a peptide of the invention via nasal inhalation in an amount that results in the T cells of said mammal having diminished capability to develop a systemic and/or peripheral immune response when they are subsequently contacted with an antigen comprising an immunodominant and/or universal portion of said peptide.

Thus, the invention provides an isolated peptide comprising a portion (fragment) of fVIII which comprises a universal immunodominant epitope sequence, e.g., any one of SEQ ID NOs:1-8, an immunogenic fragment or a variant thereof. As used herein, a “peptide” of the invention is at least 7 residues, preferably at least 10 to 20 residues, but less than 80 residues in length. These peptides are particularly useful to inhibit or prevent aberrant antibody production in disorders or diseases characterized by undesirable antibody production specific for fVIII, a biologically active fragment or a functional equivalent thereof. Thus, the invention provides a method of preventing or inhibiting aberrant, e.g., excessive, pathogenic or otherwise undesirable antibody production associated within an immune response to fVIII, a biologically active fragment thereof or a functional equivalent thereof. The method comprises administering to a mammal having, or at risk of developing, antibody inhibitors to fVIII, a biologically active fragment thereof or a functional equivalent thereof, an amount of at least one epitope peptide of fVIII or a variant thereof which peptide comprises at least one immunodominant and/or universal epitope and is effective to prevent or inhibit at least one complication of hemophilia, e.g., to reduce or decrease pathogenic antibody production or induce immune tolerance to fVIII, a biologically active fragment thereof or a functional equivalent thereof.

Also provided is a method in which the administration of a peptide of the invention to a mammal results in the suppression, tolerization, or down regulation of the priming and/or activity, of T cells of a mammal at risk of developing antibody inhibitors to fVIII, a biologically active fragment or a functional equivalent thereof, or having antibody inhibitors to fVIII, a biologically active fragment thereof or a functional equivalent thereof. Further provided is a method in which the administration of a peptide of the invention results in the decrease in the amount or activity of antibodies which are characteristic of hemophilia, i.e., fVIII inhibitors. Preferably, the administration of a peptide of the invention to a mammal results in T cell tolerization, the down regulation of priming or activity of T cells, an enhancement in the activity of or levels of modulatory T cells, and/or a reduction in the amount or affinity of pathogenic fVIII-specific antibodies.

Further provided is a method to tolerize a mammal to an antigen associated with aberrant or pathogenic, or otherwise undesirable, production of antibodies to fVIII, a biologically active fragment or functional equivalent thereof, in that mammal. In one embodiment, the method comprises administering to the mammal an amount of at least one fVIII epitope peptide, a variant thereof, or a combination thereof, having a universal and/or immunodominant epitope sequence effective to tolerize, down regulate the priming or activity of T cells of, or stimulate modulatory T cells, of the mammal to fVIII, a biologically active fragment or functional equivalent thereof.

Thus, the invention also provides a tolerogen comprising at least one isolated and purified fVIII epitope peptide having a universal and/or immunodominant epitope sequence and a physiologically compatible carrier, the administration of which to a sensitized mammal results in the suppression or reduction of the immune response of that mammal to fVIII, a biologically active fragment or functional equivalent thereof. Alternatively, the administration of at least one isolated and purified fVIII epitope peptide having a universal and/or immunodominant epitope sequence and a physiologically compatible carrier, to a non-sensitized mammal results in the blocking of or a reduction in the priming to fVIII, a biologically active fragment or functional equivalent thereof, when such antigen is administered to the mammal in a manner that normally results in an immune response. It is preferred that the peptide contains a contiguous sequence of at least about 7 amino acids having identity with the amino acid sequence of fVIII, and that the peptide is no more than about 80, preferably 60 or fewer, e.g., 40, amino acid residues in length, i.e., it represents a fragment of fVIII. It is also preferred that the tolerogen is nasally, intravenously or subcutaneously administered. In one embodiment, the peptides are co-administered with fVIII, a biologically active fragment or functional equivalent thereof, e.g., intravenously or via gene therapy.

A further embodiment of the invention is a method to inhibit or suppress the formation of antibody inhibitors of fVIII, a biologically active fragment or functional equivalent thereof, which is associated with the administration of fVIII, a biologically active fragment thereof or a functional equivalent thereof, or the use of gene therapy to replace such a protein. The fVIII, a biologically active fragment or functional equivalent thereof, may be recombinantly produced (referred to as “recombinant” protein or polypeptide), or expressed from a vector, e.g., a viral vector, for replacement gene therapy. Because fVIII, a biologically active fragment or functional equivalent thereof, is “foreign” to a mammal having hemophilia A, the mammal may have an immune response to these proteins. To suppress this response, a mammal at risk of developing antibody inhibitors to fVIII or having antibody inhibitors to fVIII, is administered a peptide of the invention, a variant thereof, or a combination thereof, in an amount effective to suppress or tolerize, stimulate modulatory T cells, or down regulate the priming and/or activity of, T cells specific for fVIII, a biologically active fragment or functional equivalent thereof.

### BRIEF DESCRIPTION OF THE FIGURES

FIG. 1. Domain structure of human factor VIII. fVIII is synthesized as a precursor of 2332 amino acids, that comprises three distinct types of domains (A1, A2, B, A3, C1 and C2). Thrombin cleavage activates the precursor, and generates the heavy chain, which consists of the A1, A2 and B domains, and the light chain, which includes the A3, C1 and C2 domains. All the A and C domains are required for the coagulant activity of fVIII, while the B domain is not. The A domains are similar in their sequence and three dimensional structure.

FIG. 2. Proliferative response to fVIII and to the individual synthetic peptides spanning the sequence of the A3 and C2 domains, of CD4⁺ splenocytes from hemophilia A mice immunized with human fVIII. The columns represent the average incorporation (±standard deviation) of the ³H-thymidine in replicate cultures, in the presence of the antigen indicated below each column. The CD4⁺ splenocytes proliferated vigorously in response to human fVIII, to an extent comparable to that observed for the non specific mitogen, PHA. The CD4⁺ splenocytes recognized several peptides, which included: on the A1 domain, the peptides spanning the sequence region 61-110; on the A2 domain, the overlapping peptides 521-540 and 531-550, and peptide 601-620; on the A3 domain, peptides 1701-1720 and 1851-1870; on the C1 domain, peptide 2131-2150; and on the C2 domain peptide 2201-2220. The stars represent statistically significant increases in the incorporation of ³H-thymidine in the presence of the antigen, as compared to the basal incorporation of cell cultures that were not exposed to any antigen.

FIG. 3. Nasal and intravenous administration of synthetic CD4⁺ epitopes of fVIII to hemophilia A mice, reduces the synthesis of anti-fVIII antibodies after exposure to fVIII intravenously. The left panel reports the results obtained in 8 mice sham-tolerized with clean PBS. The right panel reports the results obtained in 7 mice treated nasally with a pool of six synthetic sequence of human fVIII recognized by the mouse CD4+ cells sensitized to fVIII. The peptides (50 μg of each peptide) were administered nasally twice a week for three weeks before beginning the intravenous administrations of human fVIII. The control mice were treated nasally with clean PBS. After beginning treatment with fVIII, the peptides (or the clean PBS) were administered nasally once per week. Each mouse received 1 μg of fVIII intravenously every two weeks for a total of up to nine injections. The mice treated nasally with the fVIII peptides received intravenous injections of fVIII mixed with the epitope peptide pool (25 μg of each peptide in each injection). The control mice received intravenous administrations of fVIII alone. The data are the ELISA measurements of the concentration of anti-human fVIII IgG in the mouse sera. Sera from mice that had not received any treatment with fVIII or with fVIII sequences yielded values lower than 25 μg/mL. The shaded area at the bottom of each graph includes the values lower than 25 μg/mL, which should be considered as background. All mice sham tolerized with clean PBS produced substantial amounts of anti-fVIII IgG antibodies, whereas only one mouse tolerized with fVIII peptides developed consistent, albeit modest, anti-fVIII antibodies. Another two peptide-tolerized mice developed transient, minimal amounts of anti-fVIII antibodies. See text for experimental details.

FIG. 4. Recognition of individual A3 peptides by CD4⁺ blood lymphocytes from two hemophilia A patients with inhibitors. The columns represent the results of microproliferation assays, in which CD4+ blood lymphocytes were cultured in the presence of a roughly equimolar pool of all peptides spanning the sequence of the A3 domain (A3 pool; used in the cultures at a final concentration of 2 μg of each peptide) or the individual peptides spanning the sequence of the A3 domain (at a final concentration of 2 μg). The columns represent the average stimulation index (±standard deviation) of sextuplet cell cultures, cultured in the presence of the antigen indicated below the plots. The cells recognized vigorously the A3 pool, and also individual peptides. Patient # 5 recognized a richer peptide repertoire than patient # 8, that included the two peptides (1801-1820 and 1951-1970) recognized also by patient # 8. The more limited repertoire of patient # 8 might be related to the tolerance therapy with high doses of fVIII that this patient had received in the past. The intensity of the responses and the scattering of the data is representative of those obtained in all experiments in which we found a significant response to individual peptides spanning the sequence of the A3 or C2 domains.

FIG. 5. Crystallographic B factors of the sequence region of ceruloplasmin homologous to the A3 domain and universal CD4⁺ cell epitopes of the A3 domain of fVIII. The crystallographic B factors of the Ca atoms of the sequence region of ceruloplasmin homologous to the A3 domain of fVIII were plotted, as a function of the residue number in the homologous sequence of the A3 domain, starting with its amino terminal residue and ending with the carboxyl terminal residue. The B factors of the a carbon were used as they best reflect the mobility of the peptide backbone. The location of the synthetic peptide sequences of the A3 domain which were part of sequence regions that form universal CD4⁺ epitopes are indicated by lines and related residue numbers. The crystallographic data for ceruloplasmin were obtained from the Protein Data Bank.

FIG. 6. Crystallographic B factors of the carbons in the polypeptide backbone of the C2 domain and universal CD4⁺ cell epitopes. The crystallographic factors of the Ca atoms was plotted, as a function of the residue number in the sequence of the C2 domain, starting with its amino terminal residue and ending with the carboxyl terminal residue. The B factors of the a carbon were used as they best reflect the mobility of the peptide backbone. The location of the synthetic peptides of the C2 domain comprised in the sequences that form universal CD4⁺ epitopes are indicated by lines and related residue numbers. The crystallographic data for the C2 domain of human fVIII were obtained from the Protein Data Bank.

FIG. 7. Location of the universal CD4⁺ epitope sequences 1691-1710, 1801-1820, and 1941-1960 in a three dimensional structural model of the A3 domain based on the known crystal structure of ceruloplasmin. Significant portions of each of these sequence regions are located in parts of the fVIII molecule that are expected to have a high degree of solvent exposure. Also, relatively unstructured sequence loops are present in each of these sequence regions.

FIG. 8. Location of the universal CD4⁺ epitope sequences 2181-2240 and 2291-2330 within the three dimensional structure of the C2 domain. Similar to the situation observed for the A3 domain, significant portions of each of these sequence regions are exposed to the solvent, and relatively unstructured sequence loops are present in each of these sequence regions.

### DETAILED DESCRIPTION OF THE INVENTION

Definitions

“Immunodominant” CD4+ cell epitopes (also referred to as immunodominant T cell epitopes or immunodominant epitope sequences) refer to a sequence of a protein antigen, or the proteinaceous portion of an antigen, that is strongly recognized by the CD4+ cells of a mammal sensitized to that antigen, as detected by methods well known to the art, including methods described herein.

T cell epitopes can vary in size, and as few as 7 consecutive amino acid residues of a particular antigen may be recognized by CD4+ cells. Thus, an immunodominant epitope sequence is an amino acid sequence containing the smallest number of contiguous amino acid residues which are strongly recognized by T cells from an individual mammal. An epitope peptide of the invention may comprise more than one immunodominant epitope sequence, and may comprise sequences which do not contain an immunodominant epitope sequence. Sequences which do not contribute to an immunodominant epitope sequence can be present at either or both the amino- or carboxyl-terminal end of the peptide. The non-immunodominant epitope sequences preferably are no more than about 10-20 peptidyl residues in toto, and either do not affect the biological activity of the peptide or do not reduce the activity of the peptide by more than 10-20%. Preferably, epitope peptides having immunodominant epitope sequences are useful to tolerize, stimulate modulatory T cells, or down regulate the priming and/or activity of T cells of, a mammal to fVIII, a biologically active fragment or functional equivalent thereof, so as to result in a reduction in the amount or activity of antibodies to said antigen in said mammal.

As used herein, a “universal” epitope sequence is an epitope that is recognized by CD4+ cells from a majority, preferably at least about 66%, more preferably at least about 75%, of individuals within a population of a particular mammalian species that is genetically divergent at the immune response loci, e.g., at the HLA loci in humans. T cell epitopes can vary in size, and as few as 7 consecutive amino acid residues of a particular antigen may be recognized by CD4+ cells. Thus, within the scope of the invention, a universal epitope comprises an amino acid sequence containing the smallest number of contiguous amino acid residues which are recognized by CD4+ cells from a majority of mammals from the same species which are genetically different at their immune response loci. A peptide of the invention may comprise more than one universal epitope sequence, and may comprise sequences which do not contain a universal epitope sequence. Preferably, at least a majority, i.e., 51%, of the amino acid sequence of the peptide comprises a universal epitope sequence. Sequences which do not contribute to a universal epitope sequence can be present at either or both the amino- or carboxyl-terminal end of the peptide. The non-universal epitope sequences preferably are no more than about 10-20 peptidyl residues in toto, and either do not affect the biological activity of the peptide or do not reduce the activity of the peptide by more than 10-20%.

The term “tolerance” is here defined as a reduction in the T cell and/or antibody response which is specific for a given antigen. The reduction in the antibody response may be concomitant with increased sensitization and/or response of special subsets of T cells specific for the antigen, for example CD4+ Th2 or Th3 cells, or other T cell subsets, which have immunoregulatory functions.

As used herein, the terms “isolated and/or purified” refer to in vitro preparation, isolation and/or purification of a peptide or nucleic acid molecule of the invention, so that it is not associated with in vivo substances, or is substantially purified from in vitro substances.

As used herein, the term “immunogenic” with respect to a peptide of the invention means that the peptide can induce non-tolerized peripheral blood mononuclear cells (PBMC) or other lymphoid cells from a sensitized mammal to proliferate or secrete cytokines when those cells are exposed to the peptide relative to cells not exposed to the peptide, and/or that the administration of the peptide to a mammal causes an immune response to the peptide.

A “sensitized” mammal is a mammal that has been exposed to a particular antigen, as evidenced by the presence of antibodies or T cells specific to the antigen. Preferably, the mammal has high affinity, e.g., IgG, antibodies to the antigen. A sensitized mammal within the scope of the invention includes mammals having or at risk of developing antibody inhibitors to fVIII.

As used herein, an “endogenous” antigen includes proteins that are normally encoded by the genome of and expressed in a mammal.

As used herein, the term “aerosol” includes finely divided solid or liquid particles that may be created using a pressurized system such as a nebulizer or instilled into a host. The liquid or solid source material contains a peptide or a nucleic acid molecule of the invention, or a combination thereof.

An “epitope” peptide of the invention is a peptide subunit that comprises at least about 7 and no more than 80 amino acid residues which has 100% contiguous amino acid sequence homology or identity to the amino acid sequence of fVIII. An epitope peptide of the invention comprises a universal and/or immunodominant epitope sequence. The administration of an epitope peptide of the invention to a sensitized mammal results in a mammal that is tolerized to the antigen from which the epitope peptide is derived. Preferably, the administration of an epitope peptide of the invention to a mammal does not result in the stimulation of B cells specific for the peptide.

As employed herein, a “variant” of an epitope peptide of the invention refers to a peptide which comprises at least about 7 and no more than about 80, peptidyl residues which have at least about 70%, preferably about 80%, and more preferably about 90%, but less than 100%, contiguous homology or identity to the amino acid sequence of a particular antigen. A variant peptide of the invention comprises a universal and/or immunodominant epitope sequence. The administration of a variant peptide of the invention to a sensitized mammal results in a mammal that is tolerized to the peptide, and to the antigen from which the peptide is derived. Preferred variant peptides of the invention do not reduce the biological activity of the peptide by more than 10-20% relative to the corresponding non-variant peptide.

As used herein, the term “biological activity” with respect to a peptide of the invention is defined to mean that the administration of the peptide to a mammal results in the mammal developing tolerance to fVIII, a biologically active fragment or functional equivalent thereof.

“Replacement therapy” or “replacement gene therapy” as used herein means therapy intended to supplement reduced amounts or the complete absence of an endogenous protein. The replacement therapy may include the administration of isolated native protein or recombinant polypeptide, i.e., fVIII, a biologically active fragment or functional equivalent thereof, to the mammal in need thereof, or it may include the administration of a recombinant viral vector encoding fVIII, a biologically active fragment or functional equivalent thereof (“replacement gene therapy”).

I. Domain Structure of fVIII

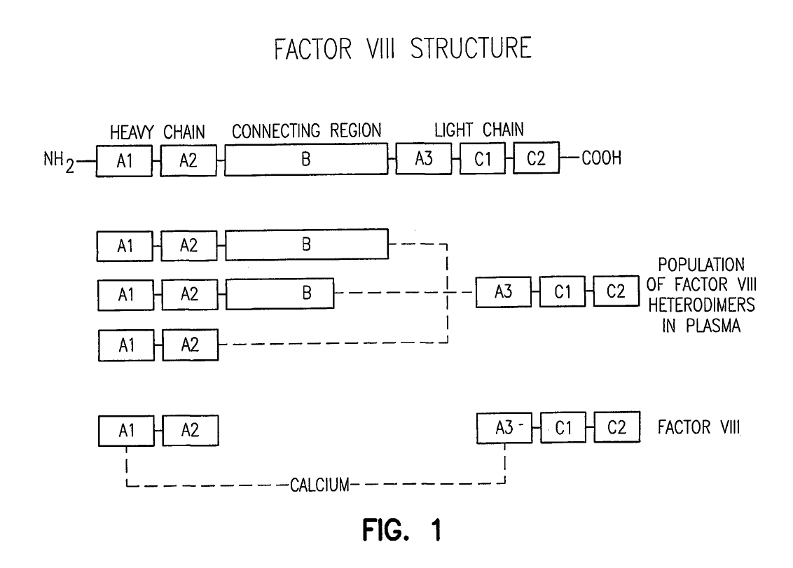

fVIII is a large glycoprotein synthesized as a precursor of 2332 amino acids, that comprises three distinct types of domains (A1, A2, B, A3, C1 and C2) (Vehar et al., 1984) (FIG. 1). These domains are usually defined by reference to thrombin cleavage sites Lollar et al., 1998). The A domains are structurally similar in their sequence and three dimensional structure (Vehar et al., 1984). Also, they are similar in their structure to other serum proteins such as ceruloplasmin (Vehar et al., 1984). Thrombin cleavage activates the precursor, and generates the heavy chain, which consists of the A1, A2 and B domains, and the light chain, which includes the A3, C1 and C2 domains (FIG. 1). All the A and C domains are required for the coagulant activity of fVIII, while the B domain is not.

Anti-fVIII antibodies are polyclonal and recognize a variety of epitopes Allain et al., 1981; Hoyer et al., 1984). Some antibodies are not inhibitory Nilsson et al., 1990; Gilles et al., 1993). Most inhibitors bind to areas of the fVIII surface on the C2, A2 and A3 domains (Lollar, 1999), that are crucial for the pro-coagulant function of fVIII. Several studies have attempted to identify the regions of the fVIII sequence and the individual residues, that form binding sites for inhibitors (Table 1: the residue numbers refer to the position, on the sequence of the fVIII precursor, of the first and last residue of the different sequence regions identified in those studies).

The synthesis of inhibitors requires CD4⁺ T helper cells: inhibitors disappear spontaneously in HIV-infected hemophiliacs, when their CD4⁺ T cell counts decline (Bray et al., 1993). Also, blockade of the B7/CD28 co-stimulatory pathway of T cell activation prevented inhibitor synthesis in a mouse model of hemophilia A (Qian et al., 1992).

About one every six healthy blood donors has low titers of anti-fVIII IgG antibody (Algiman et al., 1992; Gilles et al., 1994; Batlle et al., 1996), that sometimes inhibit fVIII in vitro Algiman et al., 1992). They recognize primarily an epitope(s) in the A3 domain (Gilles et al., 1994), in addition to several minor epitopes on the heavy chain, which includes the A1 and A2 domains (FIG. 1). Thus, healthy subjects have fVIII-specific CD4⁺ T helper cells (Reding et al., 1999). This is not surprising: healthy people have potentially autoreactive T cells, and sometimes autoreactive antibodies, against a variety of autoantigens (Bums et al., 1983; Morel-Kopp et al., 1992; Ito et al., 1993; Hoffman et al., 1993; Moiola et al., 1994; Kuwana et al., 1995).

TABLE 1
Location of fVIII inhibitor epitopes.

| Domain | Residues | Notes |
|---|---|---|
| A2 | 484-508 | Overlaps the coagulation factor X activation site |
| A3 | 1804-1819 | Overlaps part of the coagulation factor IXa |
| | | binding site |
| C2 | 2181-2243 | Overlaps the binding sites for phospholipids and |
| | | vonWillebrand factor |

Most inhibitors in hemophilia A patients are IgG4 and IgG1 (Allain et al., 1981; Hoyer et al., 1984), which are induced by Th2 and Th1 cells, respectively. In hemophilia A mice the anti-fVIII antibody are of subclasses homologous to human IgG4 and IgG1 (Wu et al., 2001). Thus, both Th1 and Th2 cells are involved in inhibitor synthesis.

II. The Immune Response

The capacity to respond to immunologic stimuli resides primarily in the cells of the lymphoid system. During embryonic life, a stem cell develops, which differentiates along several different lines. For example, the stem cell may turn into a lymphoid stem cell which may differentiate to form at least two distinct lymphoid populations. One population, called T lymphocytes, is the effector agent in cell-mediated immunity, while the other, called B lymphocytes, is the primary effector of antibody-mediated, or humoral, immunity. The stimulus for B cell antibody production is the attachment of an antigen to B cell surface immunoglobulin. Thus, B cell populations are largely responsible for specific antibody production in the host. For most antigens, B cells require the cooperation of antigen-specific T helper (CD4+) cells for effective production of high affinity antibodies.

Of the classes of T lymphocytes, T helper (Th) or CD4+ cells, are antigen-specific cells that are involved in primary immune recognition and host defense reactions against bacterial, viral, fingi and other antigens. CD4+ cells are necessary to trigger high affinity IgG production from B cells for the vast majority of antigens. The T cytotoxic (Tc) cells are antigen-specific effector cells which can kill target cells following their infection by pathologic agents.

While CD4+ cells are antigen-specific, they cannot recognize free antigen. For recognition and subsequent CD4+ activation and proliferation to occur, the antigen must be processed by suitable cells (antigen presenting cells, APC). APC fragment the antigen molecule and associate the fragments with major histocompatibility complex (MHC) class II products (in humans) present on the APC cell surface. These antigen fragments, or T cell epitopes, are thus presented to receptors or a receptor complex on the CD4+ cell in association with MHC class II products. Thus, CD4+ cell recognition of a pathogenic antigen is MHC class II restricted in that a given population of CD4+ cells must be either autologous or share one or more MHC class II products with the APC. Likewise, Tc cells recognize antigen in association with MHC class I products.

In the case of CD4+ cells, this antigen presenting function is performed by a limited number of APC. It is now well established that CD4+ cells recognize peptides derived from processed soluble antigen in association with class II MHC product, expressed on the surface of macrophages. Recently, other cell types such as resting and activated B cells, dendritic cells, epidermal Langerhans' cells, and human dermal fibroblasts have also been shown to present antigen to CD4+ T cells.

If a given CD4+ cell possesses receptors or a receptor complex which enable it to recognize a given MHC class II product-antigen complex, it becomes activated, proliferates and generates lympholines, such as interleukin 2 (IL-2).

After stimulation subsides, survivors of the expanded CD4+ cells remain as member cells in the body, and can expand rapidly again when the same antigen is presented.

Methods of determining whether PBMCs or lymphoid cells have proliferated, or produced or secreted interleukins, are well known in the art. For example, see Paul, Fundamental Immunology 3rd ed., Raven Press (1993), and Benjamini et al. (eds.), Immunology:A Short Course, John Wiley & Sons, Inc., 3rd ed. (1996).

A. Different Roles of CD4⁺ T Cell Subsets

CD4⁺ cells comprise populations that differ in their function and the cytokines they secrete (Abbas et al., 1996; Romagnani, 1997; Weigle et al., 1997; Seder et al., 1994; Constant et al., 1997). The most simple division is in Th1 and Th2 cells.

IL-12 and IFN-γ promote differentiation of naive CD4+ T cells into Th1 cells. Activated Th1 cells secrete IFN-γ, thus promoting their own proliferation and differentiation of CD4+ cells into Th1 cells. Th1 cells carry out different effector functions of the immune system. They secrete pro-inflammatory cytokines, such as IFN-γ and IL-2, and may be cytotoxic. Also, they help synthesis of IgG subclasses that bind complement, such as IgG1 in humans and IgG2 in mice.

IL-4 promotes differentiation of naive CD4⁺ T cells into Th2 cells. Activated Th2 cells secrete IL-4, and promote their own proliferation and the differentiation of naive CD4⁺ cells into Th2 cells. IL-4 inhibits Th1 cells and is a growth factor for B cells (Seder et al., 1994; Constant et al, 1997). It promotes synthesis of IgE and of IgG subclasses that do not fix complement (Seder et al., 1994; Constant et al, 1997). Th2 cells produce other cytokines, including IL-10, which is a powerful anti-inflammatory molecule (Constant et al, 1997). IL-10 inhibits development and proliferation of Th1 cells (de Waal Malefyt et al, 1993; Taga et al, 1993; Groux et al., 1996), and the function of a variety of antigen presenting cells (Ding et al., 1992; Macatonia et al., 1993; Ding et al., 1993; Enk et al., 1993). IL-10, especially in association with IL-2, is also a factor for growth and differentiation of B cells (Burdin et al., 1995; Rousset et al., 1995; Malisan et al., 1996; Kindler et al., 1997).

Thus, while Th1 cells mediate important effector functions of the immune response, by virtue of their cytotoxic ability, and by stimulating synthesis of antibody that fix complement, Th2 cells have complex and contrasting functions. They carry out effector functions by secreting IL-4 and IL-10, which stimulate growth and differentiation of B cells and help production of non-complement fixing antibody. Also, Th2 cells down regulate immune responses, by secreting anti-inflammatory cytokines, including IL-4 and IL-10, which inhibit the function of antigen presenting cells and Th1 effectors.

Th2 cells may down regulate immune responses also through the action of IL-4 on other modulatory CD4⁺ cells, that secrete TGF-β (also called Th3 cells). The TGF-β family of cytokines are potent immuno-modulators (O'Garra et al., 1997; Letterio et al., 1998) that polarize CD4⁺ responses towards a Th2 phenotype (O'Garra et al., 1997; Letterio et al., 1998; King et al., 1998) and block the effects of IL-12 in the development of Th1 responses (Letterio et al., 1998; Gorham et al., 1998; Bright et al., 1998). IL-4 is a growth factor for Th3 cells (O'Garra et al., 1997; Seder et al., 1998; Shi et al., 1999). Th3 cells do not produce IL-4, and may depend upon Th2 cells for proliferative signals (O'Garra, 1998).

B. Immune Tolerance Can Be Induced in Adult Life

Tolerance, which prevents immune responses to self-antigens, is induced and maintained by an interplay of different mechanisms. These include clonal deletion of autoreactive T cells during maturation of the immune system (Kappler et al., 1987; Schwartz, 1989), and mechanisms that operate during the adult life, such as anergy and deletion of antigen-specific T and B cells (Matzinger, 1994; Nossal, 1995). Immune tolerance is a dynamic process actively maintained throughout life, rather than one which is permanently established during the prenatal and neonatal periods (Kappler et al., 1987; Schwartz, 1989). Since synthesis of anti-fVIII antibodies depends upon the action of CD4⁺ T cells specific for fVIII (Bray et al., 1993; Reding et al., 1999; Qian et al., 1998), induction of immunologic tolerance of these T cells should be an effective mechanism to prevent inhibitor formation.

Antigen-specific tolerance can be induced by administering the antigen through routes that stimulate T cell mediated modulatory mechanisms, rather than an immune response. For example, encounter with antigens through the mucosal surfaces of the respiratory and gastrointestinal tracts can result in downregulation of CD4⁺ cells and immune tolerance to those antigens (Nossal, 1995; Mowat, 1987; Holt et al., 1989; Weiner et al., 1994; Neutra et al., 1996). This is an important protective mechanism, which guards against development of immune responses to inhaled and ingested environmental antigens throughout life. Other routes of antigen administration that favor induction of tolerance, rather than stimulation of an immune response, include the subcutaneous and intraperitoneal routes, as well as the administration of the antigen, in a soluble form, intravenously (Burstein et al., 1992; Briner et al., 1993; de Wit et al., 1993; Norman et al., 1996). These procedures have proven effective for prevention and/or treatment of CD4⁺ T cell mediated immune responses (Metzler et al., 1993; Miller et al., 1994; Al-Sabbagh et al., 1996; Wang et al., 1993; Ma et al., 1995; Wu et al., 1997; Karachunski et al., 1997).

Several mechanisms may be involved in the induction of tolerance. They include anergy or deletion by apoptosis of antigen-specific T cells, and induction of antigen-specific regulatory CD4⁺ Th2 and/or Th3 cells (Weiner et al., 1994; Chen et al., 1995). We will summarize below the salient functional characteristics of the subsets of CD4⁺ T cells that are involved in induction of antigen-specific tolerance, rather that in the driving of an antigen-specific antibody response.

Tolerance can be induced by different mechanisms, depending upon the dose of antigen given (Chen et al., 1995; Chen et al., 1996; Friedman et al., 1994; Gregerson et al., 1993). Low doses of antigen, or of CD4⁺ epitope sequences of the antigen, generate regulatory Th2 and Th3 cells (Chen et al., 1996; Friedman et al., 1994; Gregerson et al., 1993), which may exert a modulatory activity through secretion of cytokines, such as IL-4, IL-10 and TGF-β. Those cytokines that act on Th1 cells in topographic proximity, irrespective of their antigen specificity (antigen-driven bystander suppression). Also, they down regulate the activity of other cellular components of the immune system, like the antigen presenting cells and the B cells that synthesize antibodies (Weiner et al., 1994). In contrast, high doses of antigen or antigen epitopes induce anergy (Friedman et al., 1994; Gregerson et al., 1993) and/or apoptosis of antigen-reactive Th1 and Th2 cells (Chen et al., 1995; Critchfield et al., 1994). Inactivation of Th2 cells requires higher antigen doses than Th1 cell inactivation (Weiner et al., 1994; Chen et al., 1994; Chen et al., 1996; Friedman et al., 19-94; Gregerson et al., 1993; Vardhachary et al., 1997; Zhang et al., 1997), possibly because Th2 cells are resistant to activation-induced cell death mediated by Fas/FasL signaling (Vardhachary et al., 1997; Zhang et al., 1997).

C. Dangers of Antigen-specific Tolerance Induction

Nasal, oral, or systemic administration of antigens for tolerance induction have potential dangers. These procedures may stimulate antigen-specific Th2 cells that act as helper cells, and cause increased synthesis of Th2-driven antibody (Abbas et al., 1996; Neutra et al., 1996; Genain et al., 1996; O'Garra, 1998). Also, the administered antigen can stimulate specific B cells directly (Abbas et al., 1996; Neutra et al., 1996; Genain et al., 1996; Husby et al., 1994). Either case would cause formation of antigen-specific antibody, that may exacerbate the clinical condition if native antigen were used for the tolerization procedure. This may have disastrous consequences, as shown in marmoset experimental autoimmune encephalomyelitis (Genain et al., 1996) and mouse experimental myasthenia gravis (Karachunski et al., 1999). This danger would be acute in hemophilia A, because the inhibitors are frequently of IgG subclasses induced by Th2 cells: the use of fVIII for tolerization procedures would likely result in antibody that would exacerbate, rather than alleviate, the problem.

Short, denatured peptide sequences of the antigen, that form epitopes recognized by the antigen-specific CD4⁺ cells are much safer than the whole antigen for T cell tolerance procedures, because their use would lead to the formation of antibody specific for the peptide(s) used. Peptide-specific antibody crossreact seldom with the cognate native antigen (Conti-Fine et al., 1996), and should not have deleterious effects.

Tolerance induced by feeding large amounts of antigen has been successfully used in a variety of experimental autoimmune responses, including antibody-mediated experimental autoimmune diseases (Weiner et al., 1994; Chen et al., 1995; Chen et al., 1996; Friedman et al., 1994; Gregerson et al., 1993; Liblau et al., 1995; Miller et al, 1994; Chen et al., 1994; Weiner, 1997; von Herrath et al., 1996; Chen et al., 1996). It has been attempted in some human autoimmune diseases such as multiple sclerosis, by feeding bovine myelin, and in rheumatoid arthritis, by feeding chicken collagen (Trentham et al., 1993; Weiner et al., 1993). These treatments resulted in neither adverse reactions nor therapeutic benefit. These outcomes are probably explained by the fact that the gastrointestinal tract is a proteolytic barrier which cannot be penetrated by intact protein antigens. CD4⁺ epitope peptides are especially ill suited for oral tolerance (Metzler et al., 1993), because short denatured sequences are exceedingly easy targets for proteases.

In nasal, subcutaneous or intravenous tolerance procedures there is no need to overcome proteolytic barriers. Thus, small amounts of short synthetic sequences forming CD4⁺ epitopes can be used (Karachunski et al., 1999; Metzler et al., 1993; Karachunski et al, 1997; Wu et al., 1997). Peptides are even more effective than the whole antigen, because their small size facilitates their diffusion (Metzler et al., 1993). Nasal or subcutaneous administration to mice of synthetic acetylcholine receptor sequences forming CD4⁺ epitopes caused CD4⁺ cells to become unresponsive to those epitopes, and prevented the synthesis of anti-receptor antibody and the induction of experimental myasthenia gravis (Karachunski et al., 1999; Karachunski et al., 1997; Wu et al., 1997).

Thus, it is possible to induce epitope-specific tolerization of CD4⁺ cells, thereby suppressing the synthesis of specific, pathogenic antibody.

D. Human CD4⁺ Cells Recognize Universal Epitope Sequences

A finding that has important ramifications for development of tolerization procedures to fVIII is the demonstration that in humans a few small regions of the sequence of an antigen may be recognized by the CD4⁺ cells of every subject, irrespective of their HLA-class II haplotype (Panina-Bordignon et al., 1989; Ho et al., 1990; Reece et al., 1993; Protti et al., 1993; Raju et al., 1995; Diethelm et al., 1997; Wang et al., 1997). These regions have been termed “universal CD4⁺ epitopes”. They are also immunodominant, in the sense that they are able to sensitize a large number of CD4⁺ cells (Raju et al., 1995; Diethelm et al., 1997; Wang et al., 1997). The presence of universal CD4⁺ epitopes has been demonstrated on both self-antigens, like the muscle acetylcholine receptor (Protti et al., 1993; Wang et al., 1997), and on foreign antigens, like diphtheria toxoid (DTD) (Raju et al., 1995), and tetanus toxoid (TTD) (Panina-Bordignon et al., 1989; Ho et al., 1990; Reece et al., 1993; Dietheim et al., 1997).

The sequence regions that flank a T epitope may modulate its immunogenicity (Moudgil et al., 1998). This night be due to structural properties that facilitate proteolytic cleavage: the sequences most effective at sensitizing CD4⁺ cells may be those easily processed and released from the antigen (Raju et al, 1995). This, and the promiscuous peptide binding of human class II molecules (Watts, 1997; Madden, 1995; Cresswell, 1994), may result in their universal recognition.

While the three dimensional structure of the nicotinic acetylcholine receptor is not known, the crystal structure of DTD has been solved (Choe et al. 1992). Part of the three dimensional structure of TTD has been solved (Umland et al., 1997), and the remainder part of the TTD molecule has been modeled, based on the known three dimensional structure of a highly similar toxin, botulinum toxin (Lacy et al., 1998). This has permitted us to identify three dimensional structural features of the different parts of these molecules that correlate with the presence of immunodominant, universal epitopes (Raju et al., 1995; Diethelm-Okita et al., 2000). Universal CD4⁺ epitopes identified on DTD and TTD all included, or were flanked by, residues forming loops fully exposed to the solvent (Raju et al., 1995; Dietheln-Okita et al., 2000). Such loops would be easy targets for the proteases involved in antigen processing. Also, universal CD4⁺ epitopes all aligned with parts of the TTD and DTD sequences which likely have low atomic mobility, as determined in crystallographic studies (Choe et al., 1992; Umland et al., 1997; Lacy et al., 1998), and they were flanked by sequence segments with high atomic mobility. Flexible, solvent-exposed loops would be ideal targets of processing proteases, and their presence may facilitate the CD4⁺ immunodominance of the intervening sequence. Thus, the presence of solvent-exposed, mobile sequence loops at both ends of a sequence region are good predictors of a universal epitope for human CD4⁺ cells.

m. Identification and Preparation of an Epitope Peptide of the Invention

A. Identification

The identification of a universal and/or immunodominant epitope sequence in an antigen permits the development and use of a peptide-based tolerogen to the antigen. The administration of epitope peptides which contain a universal and/or immunodominant epitope sequence can induce a tolerizing effect in many, if not all, mammals, preferably those of differing immune response haplotypes. Moreover, the use of peptide tolerogens is less likely to produce the undesirable side effects associated with the use of the full-length antigen. These epitope peptides can be identified by in vitro and in vivo assays, such as the assays described hereinbelow (see, for example, Conti-Fine et al., 1997; and Wang et al., 1997). It is recognized that not all agents falling within the scope of the invention can result in tolerization, or result in the same degree of tolerization.

To identify epitope peptides useful to tolerize a mammal having or at risk of an indication or disease within the scope of the invention, the antigen which is associated with the indication or disease is identified. The antigen may be fVIII, or a biologically active fragment or functional equivalent thereof, which is administered exogenously to a mammal to correct a deficiency in that protein or synthesized from a vector that is administered to the mammal for replacement gene therapy. Generally, 20 residue peptides are obtained or prepared which span the entire amino acid sequence of the antigen and which overlap the adjacent peptide by 5-10 residues. In this manner, a peptide may include sequences which correspond to a portion of a universal and/or immunodominant epitope sequence. These peptides are then individually screened in vitro and in vivo.

In vitro methods useful to determine whether a particular peptide comprises a universal and/or immunodominant epitope sequence include determining the biological activity (e.g., inducing the proliferation of or cytokine secretion by T cells) of the peptide in CD4+ cell lines that are specific for an antigen having the peptide, isolated CD4+ cells, CD8+ depleted spleen or lymph node cells, or CD8+ depleted peripheral blood mononuclear cells (PBMC). These cells may be obtained from a mammal at risk or of having an indication or disease within the scope of the invention or from a mammal that is “normal”. In either case, the mammal is preferably known to be sensitized to the antigen. Epitope peptides useful in the practice of the invention include a peptide that is strongly recognized by the T cells of the mammal tested, i.e., they have an immunodominant epitope sequence. Preferred epitope peptides are those which are recognized by the T cells of at least a majority of mammals having divergent immune response haplotypes, e.g., MHC class II molecules in humans. This recognition can be measured by the ability of the peptide to induce proliferation or cytokine secretion in T cells obtained from mammals with known or suspected divergent haplotypes and/or by direct HLA class II binding assays (Manfredi et al., 1994; Yuen et al., 1996).

Thus, CD8+ depleted PBMC, CD8+ depleted spleen or lymph node cells or CD4+ lines specific for an antigen or epitope can be contacted with an epitope peptide and the proliferation of the cells measured or the amount and type of cytokine secreted detected. Th1 cytokines include IFN-γ, IL-12 and IL-2. Th2 cytokines include IL-4 and IL-10. An immunospot ELISA or other biological assay is employed to determine the cytokine which is secreted after the peptide is added to the culture.

Epitope peptides falling within the scope of the invention may also be identified by in vivo assays, such as animal models for hemophilia A.

B. Preparation of the Nucleic Acid Molecules of the Invention

1. Sources of the Nucleic Acid Molecules of the Invention

Sources of nucleotide sequences from which a nucleic acid molecule encoding a fVIII peptide or variant thereof of the invention, or a variant thereof, include total or polyA⁺ RNA from any eukaryotic, preferably mammalian, cellular source from which cDNAs can be derived by methods known in the art. Other sources of DNA molecules of the invention include genomic libraries derived from any eukaryotic cellular source.

Sources of nucleotide sequences of viral vectors useful in gene therapy include RNA or DNA from virally-infected cells, plasmids having DNA encoding viral proteins, nucleic acid in viral particles and the like.

Moreover, the present DNA molecules may be prepared in vitro, e.g., by synthesizing an oligonucleotide of about 100, preferably about 75, more preferably about 50, and even more preferably about 40, nucleotides in length, or by subcloning a portion of a DNA segment that encodes a particular peptide.

2. Isolation of a Gene Encoding a Peptide of the Invention

A nucleic acid molecule encoding a peptide of the invention can be identified and isolated using standard methods, as described by Sambrook et al., (1989). For example, reverse-transcriptase PCR (RT-PCR) can be employed to isolate and clone a preselected cDNA. Oligo-dT can be employed as a primer in a reverse transcriptase reaction to prepare first-strand cDNAs from isolated RNA which contains RNA sequences of interest, e.g., total RNA isolated from human tissue. RNA can be isolated by methods known to the art, e.g., using TRIZOL reagent (GIBCO-BRL/Life Technologies, Gaithersburg, Md.). Resultant first-strand cDNAs are then amplified in PCR reactions.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers comprising at least 7-8 nucleotides. These primers will be identical or similar in sequence to opposite strands of the template to be amplified. PCR can be used to amplify specific RNA sequences, specific DNA sequences from total genomic DNA, and cDNA transcribed from total cellular RNA, bacteriophage or plasmid sequences, and the like. See generally Mullis et al., (1987); Erlich (1989). Thus, PCR-based cloning approaches rely upon conserved sequences deduced from alignments of related gene or polypeptide sequences.

Primers are made to correspond to highly conserved regions of polypeptides or nucleotide sequences which were identified and compared to generate the primers, e.g., by a sequence comparison of a particular eukaryotic gene. One primer is prepared which is predicted to anneal to the antisense strand, and another primer prepared which is predicted to anneal to the sense strand, of a nucleic acid molecule which encodes the preselected peptide.

The products of each PCR reaction are separated via an agarose gel and all consistently amplified products are gel-purified and cloned directly into a suitable vector, such as a known plasmid vector. The resultant plasmids are subjected to restriction endonuclease and dideoxy sequencing of double-stranded plasmid DNAs. Alternatively, isolated gel-purified fragments may be directly sequenced.

As used herein, the terms “isolated and/or purified” refer to in vitro isolation of a DNA, peptide or polypeptide molecule from its natural cellular environment, and from association with other components of the cell, such as nucleic acid or polypeptide, so that it can be sequenced, replicated, and/or expressed. For example, an “isolated, preselected nucleic acid” is RNA or DNA containing greater than 9, preferably 36, and more preferably 45 or more, sequential nucleotide bases that encode at least a portion of a peptide of the invention, or a variant thereof, or a RNA or DNA complementary thereto, that is complementary or hybridizes, respectively, to RNA or DNA encoding the peptide, or polypeptide having said peptide, and remains stably bound under stringent conditions, as defined by methods well known in the art, e.g., in Sambrook et al., supra. Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell. An example of an isolated nucleic acid molecule of the invention is RNA or DNA that encodes human fVIII, or a fragment or subunit thereof, and shares at least about 80%, preferably at least about 90%, and more preferably at least about 95%, contiguous sequence identity with the human fVIII polypeptide.

As used herein, the term “recombinant nucleic acid” or “preselected nucleic acid,” e.g., “recombinant DNA sequence or segment” or “preselected DNA sequence or segment” refers to a nucleic acid, e.g., to DNA, that has been derived or isolated from any appropriate tissue source, that may be subsequently chemically altered in vitro, so that its sequence is not naturally occurring, or corresponds to naturally occurring sequences that are not positioned as they would be positioned in a genome which has not been transformed with exogenous DNA. An example of preselected DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. See Lawn et al. (1981), and Goeddel et al. (1980). Therefore, “preselected DNA” includes completely synthetic DNA sequences, semi-synthetic DNA sequences, DNA sequences isolated from biological sources, and DNA sequences derived from RNA, as well as mixtures thereof.

As used herein, the term “derived” with respect to a RNA molecule means that the RNA molecule has complementary sequence identity to a particular DNA molecule.

3. Variants of the Nucleic Acid Molecules of the Invention

Nucleic acid molecules encoding amino acid sequence variants of a peptide of the invention are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the preselected peptide.

Oligonucleotide-mediated mutagenesis is a preferred method for preparing amino acid substitution variants of a peptide. This technique is well known in the art as described by Adelman et al. (1983). Briefly, DNA is altered by hybridizing an oligonucleotide encoding the desired mutation to a DNA template, where the template is the single-stranded form of a plasmid or bacteriophage containing the unaltered or native DNA sequence. After hybridization, a DNA polymerase is used to synthesize an entire second complementary strand of the template that will thus incorporate the oligonucleotide primer, and will code for the selected alteration in the preselected DNA.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (1978).

The DNA template can be generated by those vectors that are either derived from bacteriophage M13 vectors (the commercially available M13mp18 and M13mp19 vectors are suitable), or those vectors that contain a single-stranded phage origin of replication as described by Viera et al. (1987). Thus, the DNA that is to be mutated may be inserted into one of these vectors to generate single-stranded template. Production of the single-stranded template is described in Sections 4.21-4.41 of Sambrook et al. (1989).

Alternatively, single-stranded DNA template may be generated by denaturing double-stranded plasmid (or other) DNA using standard techniques.

For alteration of the native DNA sequence (to generate amino acid sequence variants, for example), the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of the peptide, and the other strand (the original template) encodes the native, unaltered sequence of the peptide. This heteroduplex molecule is then transformed into a suitable host cell, usually a prokaryote such as E. Coli JM101. After the cells are grown, they are plated onto agarose plates and screened using the oligonucleotide primer radiolabeled with 32-phosphate to identify the bacterial colonies that contain the mutated DNA The mutated region is then removed and placed in an appropriate vector for peptide or polypeptide production, generally an expression vector of the type typically employed for transformation of an appropriate host.

The method described immediately above may be modified such that a homoduplex molecule is created wherein both strands of the plasmid contain the mutations(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(αS) (which can be obtained from the Amersharn Corporation). This mixture is added to the template-oligonucleotide complex. Upon addition of DNA polymerase to this mixture, a strand of DNA identical to the template except for the mutated bases is generated. In addition, this new strand of DNA will contain dCTP-(αS) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101.

Nucleotide substitutions can be introduced into DNA segments by methods well known to the art. See, for example, Sambrook et al., supra. Likewise, nucleic acid molecules encoding other mammalian, preferably human, or viral, peptides may be modified in a similar manner, so as to yield nucleic acid molecules of the invention having silent nucleotide substitutions, or to yield nucleic acid molecules having nucleotide substitutions that result in amino acid substitutions (see peptide variants hereinbelow).

4. Chimeric Expression Cassettes

To prepare expression cassettes for transformation herein, the recombinant or preselected DNA sequence or segment may be circular or linear, double-stranded or single-stranded. Generally, the preselected DNA sequence or segment is in the form of chimeric DNA, such as plasmid DNA, that can also contain coding regions flanked by control sequences which promote the expression of the preselected DNA present in the resultant cell line.

As used herein, “chimeric” means that a vector comprises DNA from at least two different species, or comprises DNA from the same species, which is linked or associated in a manner which does not occur in the “native” or wild type of the species.

Aside from preselected DNA sequences that serve as transcription units for a peptide, or portions thereof, a portion of the preselected DNA may be untranscribed, serving a regulatory or a structural function. For example, the preselected DNA may itself comprise a promoter that is active in mammalian cells, or may utilize a promoter already present in the genome that is the transformation target. Such promoters include the CMV promoter, as well as the SV40 late promoter and retroviral LTRs (long terminal repeat elements), although many other promoter elements well known to the art may be employed in the practice of the invention.

Other elements functional in the host cells, such as introns, enhancers, polyadenylation sequences and the like, may also be a part of the preselected DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

“Control sequences” is defined to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

“Operably linked” is defined to mean that the nucleic acids are placed in a functional relationship with another nucleic acid sequence. For example, DNA for a presequence or secretory leader is operably linked to DNA for a peptide or polypeptide if it is expressed as a preprotein that participates in the secretion of the peptide or polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The preselected DNA to be introduced into the cells further will generally contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Preferred genes include the chloramphenicol acetyl transferase gene (cat) from Tn9 of E. coli, the beta-glucuronidase gene (gus) of the uidA locus of E. coli, and the luciferase gene from firefly Photinus pyralis. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein. For example, Sambrook et al. (1989), provides suitable methods of construction.

5. Transformation into Host Cells

The recombinant DNA can be readily introduced into the host cells, e.g., mammalian, bacterial, yeast or insect cells by transfection with an expression vector comprising DNA encoding a preselected peptide by any procedure useful for the introduction into a particular cell, e.g., physical or biological methods, to yield a transformed cell having the recombinant DNA stably integrated into its genome, so that the DNA molecules, sequences, or segments, of the present invention are expressed by the host cell.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. The main advantage of physical methods is that they are not associated with pathological or oncogenic processes of viruses. However, they are less precise, often resulting in multiple copy insertions, random integration, disruption of foreign and endogenous gene sequences, and unpredictable expression. For mammalian gene therapy, it is desirable to use an efficient means of precisely inserting a single copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like. See, for example, U.S. Pat. Nos. 5,350,674 and 5,585,362.

As used herein, the term “cell line” or “host cell” is intended to refer to well-characterized homogenous, biologically pure populations of cells. These cells may be eukaryotic cells that are neoplastic or which have been “immortalized” in vitro by methods known in the art, as well as primary cells, or prokaryotic cells. The cell line or host cell is preferably of mammalian origin, but cell lines or host cells of non-mammalian origin may be employed, including plant, insect, yeast, fungal or bacterial sources. Generally, the preselected DNA sequence is related to a DNA sequence which is resident in the genome of the host cell but is not expressed, or not highly expressed, or, alternatively, overexpressed.

“Transfected” or “transformed” is used herein to include any host cell or cell line, the genome of which has been altered or augmented by the presence of at least one preselected DNA sequence, which DNA is also referred to in the art of genetic engineering as “heterologous DNA,” “recombinant DNA,” “exogenous DNA,” “genetically engineered,” “non-native,” or “foreign DNA,” wherein said DNA was isolated and introduced into the genome of the host cell or cell line by the process of genetic engineering. The host cells of the present invention are typically produced by transfection with a DNA sequence in a plasmid expression vector, a viral expression vector, or as an isolated linear DNA sequence. Preferably, the transfected DNA is a chromosomally integrated recombinant DNA sequence, which comprises a gene encoding the peptide, which host cell may or may not express significant levels of autologous or “native” polypeptide.

To confirm the presence of the preselected DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described hereinabove to identify agents falling within the scope of the invention.

To detect and quantitate RNA produced from introduced preselected DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the peptide products of the introduced preselected DNA sequences or evaluating the phenotypic changes brought about by the expression of the introduced preselected DNA segment in the host cell.

C. Peptides, Peptide Variants, and Derivatives Thereof

The present isolated, purified peptides or variants thereof, can be synthesized in vitro, e.g., by the solid phase peptide synthetic method or by recombinant DNA approaches (see above). The solid phase peptide synthetic method is an established and widely used method, which is described in the following references: Stewart et al. (1969); Merrifield (1963); Meienhofer (1973); and Bavaay and Merrifield (1980). These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, derivatives, e.g., chemically derived derivatives, of a given peptide can be readily prepared. For example, amides of the peptide or peptide variants of the present invention may also be prepared by techniques well known in the art for converting a carboxylic acid group or precursor to an amide. A preferred method for amide formation at the C-terminal carboxyl group is to cleave the peptide from a solid support with an appropriate amine, or to cleave in the presence of an alcohol, yielding an ester, followed by aminolysis with the desired amine.

Salts of carboxyl groups of a peptide or peptide variant of the invention may be prepared in the usual manner by contacting the peptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the peptide or peptide variants may be prepared by utilizing an N-acyl protected amino acid for the final condensation, or by acylating a protected or unprotected peptide. O-acyl derivatives may be prepared, for example, by acylation of a free hydroxy peptide or peptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the peptide or peptide variant. Other amino-terminal modifications include aminooxypentane modifications (see Simmons et al. (1997)).

In addition, the amino acid sequence of a peptide can be modified so as to result in a peptide variant (see above). The modification includes the substitution of at least one amino acid residue in the peptide for another amino acid residue, including substitutions which utilize the D rather than L form, as well as other well known amino acid analogs. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoyl-phenylalanine, phenylglycine, propargylglycine, sarcosine, and tert-butylglycine.

One or more of the residues of the peptide can be altered, so long as the peptide variant is biologically active. For example, it is preferred that the variant has at least about 10% of the biological activity of the corresponding non-variant peptide. Conservative amino acid substitutions are preferred—that is, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic; trp, tyr, phe.

The invention also envisions peptide variants with non-conservative substitutions. Non-conservative substitutions entail exchanging a member of one of the classes described above for another.

Acid addition salts of the peptide or variant peptide, or of amino residues of the peptide or variant peptide, may be prepared by contacting the peptide or amine with one or more equivalents of the desired inorganic or organic acid, such as, for example, hydrochloric acid. Esters of carboxyl groups of the peptides may also be prepared by any of the usual methods known in the art.

IV. Dosages, Formulations and Routes of Administration of the Peptides of the Invention

The peptides or nucleic acid molecules of the invention, including their salts, are preferably administered so as to achieve a decrease, reduction or elimination in the amount of antibody inhibitors to fVIII, a biologically active fragment or functional equivalent thereof. To achieve this effect(s), the peptide, a variant thereof or a combination thereof, agent may be administered at dosages of at least about 0.001 to about 100 mg/kg, more preferably about 0.01 to about 10 mg/kg, and even more preferably about 0.1 to about 5 mg/kg, of body weight, although other dosages may provide beneficial results. The amount administered will vary depending on various factors including, but not limited to, the agent chosen, the disease, the weight, the physical condition, and the age of the mammal, whether prevention or treatment is to be achieved, and if the agent is chemically modified. Such factors can be readily determined by the clinician employing animal models or other test systems which are well known to the art.

Administration of sense nucleic acid molecule may be accomplished through the introduction of cells transformed with an expression cassette comprising the nucleic acid molecule (see, for example, WO 93/02556) or the administration of the nucleic acid molecule (see, for example, Felgner et al., U.S. Pat. No. 5,580,859, Pardoll et al. (1995); Stevenson et al. (1995); Molling (1997); Donnelly et al. (1995); Yang et al. (1996); Abdallah et al. (1995)). Pharmaceutical formulations, dosages and routes of administration for nucleic acids are generally disclosed, for example, in Felgner et al., supra.

Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

To prepare the composition, peptides are synthesized or otherwise obtained, purified and then lyophilized and stabilized. The peptide can then be adjusted to the appropriate concentration, and optionally combined with other agents. The absolute weight of a given peptide included in a unit dose of a tolerogen can vary widely. For example, about 0.01 to about 10 mg, preferably about 0.5 to about 5 mg, of at least one peptide of the invention, and preferably a plurality of peptides specific for a particular antigen, each containing a universal and/or immunodominant epitope sequence, can be administered. A unit dose of the tolerogen is preferably administered either via a mucous membrane, e.g., by respiratory, e.g., nasal (e.g., instill or inhale aerosol), intravenously, or orally, although other routes, such as subcutaneous and intraperitoneal are envisioned to be useful to induce tolerance.

Thus, one or more suitable unit dosage forms comprising the therapeutic agents of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including oral, or parenteral, including by rectal, transdermal, subcutaneous, intravenous, intramuscular, intraperitoneal, intrathoracic, intrapulmonary and intranasal (respiratory) routes. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are preferably combined with a pharmaceutically acceptable carrier, diluent or excipient to form a pharmaceutical formulation, or unit dosage form. Preferably, orally administered therapeutic agents of the invention are formulated for sustained release, e.g., the agents are microencapsulated. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Pharmaceutical formulations containing the therapeutic agents of the invention can be prepared by procedures known in the art using well known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, BPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and α-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosage forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like.

The therapeutic agents of the invention can be delivered via patches for transdermal administration. See U.S. Pat. No. 5,560,922 for examples of patches suitable for transdermal delivery of a therapeutic agent. Patches for transdernal delivery can comprise a backing layer and a polymer matrix which has dispersed or dissolved therein a therapeutic agent, along with one or more skin permeation enhancers. The backing layer can be made of any suitable material which is impermeable to the therapeutic agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which therapeutic agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the therapeutic agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydrogel), polyvinylidene chloride, poly(ethylene terephthalate), butyl rubber, epichlorohydrin rubbers, ethylenvinyl alcohol copolymers, ethylene-vinyloxyethanol copolymers; silicone copolymers, for example, polysiloxane-polycarbonate copolymers, polysiloxanepolyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the therapeutic agent into the polymer. Known cross-linking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Therapeutic agents released from a transdermal delivery system must be capable of penetrating each layer of skin. In order to increase the rate of permeation of a therapeutic agent, a transdermal drug delivery system must be able in particular to increase the permeability of the outermost layer of skin, the stratum corneum, which provides the most resistance to the penetration of molecules. The fabrication of patches for transdermal delivery of therapeutic agents is well known to the art.

For topical administration, the therapeutic agents may be formulated as is known in the art for direct application to a target area. Conventional forms for this purpose include wound dressings, coated bandages or other polymer coverings, ointments, creams, lotions, pastes, jellies, sprays, and aerosols. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

Preferably, the peptide or nucleic acid of the invention is administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific indication or disease. Any statistically significant attenuation of one or more symptoms of an indication or disease that has been treated pursuant to the method of the present invention is considered to be a treatment of such indication or disease within the scope of the invention.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Examples of such substances include normal saline solutions such as physiologically buffered saline solutions and water.

A preferred route of administration of the therapeutic agents of the present invention is in an aerosol or inhaled form. The agents of the present invention can be administered as a dry powder or in an aqueous solution.

Preferred aerosol pharmaceutical formulations may comprise, for example, a physiologically acceptable buffered saline solution containing between about 0.1 mg/ml and about 100 mg/ml of one or more of the agents of the present invention specific for the indication or disease to be treated.

Dry aerosol in the form of finely divided solid peptide or nucleic acid particles that are not dissolved or suspended in a liquid are also useful in the practice of the present invention. Peptide or nucleic acid may be in the form of dusting powders and comprise finely divided particles having an average particle size of between about 1 and 5 μm, preferably between 2 and 3 μm. Finely divided particles may be prepared by pulverization and screen filtration using techniques well known in the art. The particles may be administered by inhaling a predetermined quantity of the finely divided material, which can be in the form of a powder.

Specific non-limiting examples of the carriers and/or diluents that are useful in the pharmaceutical formulations of the present invention include water and physiologically acceptable buffered saline solutions such as phosphate buffered saline solutions pH 7.0-8.0.

For administration to the upper (nasal) or lower respiratory tract by inhalation, the therapeutic agents of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator, or a metered-dose inhaler (see, for example, the pressurized metered dose inhaler (MDI) and the dry powder inhaler disclosed in Newman (1984).

Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.).

For intra-nasal administration, the therapeutic agent may be administered via nose drops, a liquid spray, such as via a plastic bottle atomizer or metered-dose inhaler. Typical of atomizers are the Mistometer (Wintrop) and the Medihaler (Riker).

The formulations and compositions described herein may also contain other ingredients such as antimicrobial agents, or preservatives. Furthermore, the active ingredients may also be used in combination with other therapeutic agents, for example, bronchodilators.

V. Managment and Prevention of fVIII Antibody Inhibitors

Moreover, to enhance the efficacy of peptide-based tolerance therapies for the treatment or prevention of antibodies inhibitors to fVIII, a biologically active fragment or functional equivalent thereof, plasmapheresis may be used in combination with the peptide treatment. Plasmapheresis “clears” the antibodies from the patient's blood, and it is in most cases associated with the administration of an immunosuppressant such as azathioprine, to help decrease the activity of the pathogenic immune cells. Thus, the administration of a peptide of the invention in combination with pheresis and optionally an immunosuppressant may be useful to manage both hemophilia A and acquired hemophilia as such a method would result in a long lasting down regulation of the anti-fVIII response, in both the CD4+ and the B cell compartments.

Moreover, the existence of universal CD4+ epitopes on the fVIII molecule would allow the use of these approaches for the prevention of inhibitor development. Furthermore, the identification of universal CD4+ epitope sequences for factor VIII would allow their use for tolerization procedures that would be suitable both in the treatment of established fVIII inhibitors and in the prevention of inhibitor development, by tolerizing or down regulating the priming and/or activity of the T helper clones potentially reactive to factor VIII sequences, prior to the first therapeutic exposure to factor VIII in infancy.

The invention will be further described by, but is not limited to, the following examples.

### EXAMPLE I

### Characterization of Factor VIII-Specific Immune Response in Humans

Approximately 25% of patients with severe hemophilia A develop blocking antibodies (inhibitors) to the missing coagulation factor, factor VIII (fVIII). Inhibitors block fVIII activity and significantly compromise the ability to achieve therapeutic homeostasis during bleeding episodes. fVIII inhibitors also develop also during autoimmune hemophilia A, a rare but frequently fatal disease in which fVIII is the target of autoimmune response. Hemophilia A results from a genetic defect in the fVIII gene while acquired (autoimmune) hemophilia is the result of an autoimmune response to fVIII. fVIII inhibitors are high affinity IgG. Their synthesis requires the action of CD4+ T helper cells specific for fVIII.

Healthy humans have recurrent, transient sensitization of CD4+ cells to fVIII. This is likely due to extravasation of fVIII at sites, such as bruises, where fVIII sequence may be presented by professional antigen presenting cells, able to prime potentially autoreactive CD4+ cells specific for fVIII epitopes. In normal individuals, who have high blood levels of fVIII, the activated anti-fVIII CD4+cells quickly disappear, possibly as a result of anergy or deletion by peripheral mechanisms of tolerance. Such cells persist in hemophilia A patients because their low fVIII levels, even after therapy, do not suffice for tolerization. Thus, the presence of anti-fVIII CD4+ cells in healthy humans can assist in the identification of universal CD4+ epitopes for fVIII.

Materials and Methods

Peptides. A panel of about 240 synthetic peptides, 20 residues long and overlapping by 10 residues, spanning the fVIII sequence, was screened on T cells to determine which peptides have universal and/or immunodominant epitope sequences. The peptide length compares with that of naturally processed class II restricted epitope peptides, that are 9-14 residues long (Rudensky et al., 1991; Hunt et al., 1992; Stem et al., 1994). Extra residues at either end of the epitope sequence do not affect the attachment to the binding cleft of the DR molecules, which is open at both its ends (Hunt et al., 1992; Stern et al., 1994). The ten residue overlap reduces the risk of missing epitopes “broken” between peptides.

The peptides synthesized are 70-85% pure (Houghton, 1985; Protti et al., 1990; Protti et al., 1990; Manfredi et al., 1992). Contaminants are a mixture of shorter analogs in which one or more residues are missing randomly, due to incomplete coupling. The analogs might bind the restricting class II molecule, but not the specific TCR in a manner conducive to measurable T cell response. This would result in a shift of the dose dependence of the CD4+ cell responses to the peptide, towards higher doses than when using purified peptides. Because the doses used to test human and mouse anti-fVIII CD4+ cells are generous, the risk of missing detection of the response to a peptide because of the presence of contaminating analogs is negligible.

The sequence and purity of several peptides have been checked, selected randomly, by determination of their amino acid composition (Henrickson et al., 1983) and mass-spec determination of the molecular weight of the species present in the peptide preparation. Amino acid composition analysis yielded results corresponding to the theoretical values for all peptides. Mass-spec analysis consistently yielded a major peak with the molecular weight calculated for the peptide. Further purification can be carried out by reverse phase HPLC.

Assays. The T cells are obtained from hemophilia A patients, autoimmune hemophilia patients, and healthy individuals that have a CD4+response to fVIII. Identification of the CD4+ epitope repertoire on fVIII recognized by the patients or healthy individuals can be accomplished by using at least one of three sets of complimentary experiments, as follows: 1) identification of the epitope repertoire of unselected CD4+ cells from the patient's blood by proliferation experiments using CD8+ depleted, CD4+ enriched peripheral blood lymphocytes (PBL), challenged with each individual peptide; 2) identification of the CD4+ subset (Th1 or Th2) recognizing the different fVIII epitopes, by immunospot assays of the cytokines secreted by individual blood CD4+ cells in response to challenge with the difference fVIII peptides (preferably, IL-2 and y-interferon are employed to detect Th1 cells, and IL-4 is employed to detect Th2 cells); and 3) propagation of fVIII-specific CD4+ lines, by cycles of stimulation in vitro of the PBL with fVIII followed by IL-2 or IL-4, and determination of their epitope repertoire and the Th1 or Th2 subset involved in the anti-epitope response, by challenging them with individual synthetic sequences in proliferation and immunospot assays.

To identify the CD4+ epitope repertoire on fVIII in the hemophilia A mice (Bi et al., 1995), CD8+ depleted, CD4+ enriched spleen cells are employed instead of PBL. The mice have been injected with fVIII i.v. three times prior to spleen cell isolation, or by other routes that result in an immune response to fVIII. Alternatively, CD4+ cells are purified from the spleen and reconstituted with autologous antigen presenting cells.

Results

Healthy Subjects Response to fVIII Peptide Pools. The CD4+ cells from twelve healthy subjects were screened with a pool of fVIII peptides, e.g, 24 pools of 10 peptides each. All subjects recognized one or more peptide pools. The pools comprising the sequence of the A2, A3 and C2 domains were recognized most strongly and most frequently. Anti-fVIII antibodies, including the inhibitors in hemophilia patients recognize primarily (but not exclusively) epitopes formed by the A2 and C2 domains. Thus, it appears that those domains may dominate both the pathogenic immune response to fVIII that leads to inhibitor formation in hemophilia A and the ephemeral, nonpathogenic responses of healthy subjects. Some subjects did not have a detectable response to the complete fVIII molecule, in spite of their significant response to one or more peptide pools. This is likely due to the much higher concentration of epitope sequences in the assays carried out with the peptides, than in those testing the response to fVIII.

To further investigate the response of CD4+ cells, two approaches were used. One approach utilized pools of synthetic peptides spanning the sequence of individual fVIII domains, referred to as “fVIII domain pools”. For the B domain, which is much longer than the others, two pools are used, corresponding to the amino terminal and carboxyl terminal halves of the B domain. In the second approach, the synthetic fVIII sequences are grouped in 24 pools of about 10 peptides each, starting with the amino terminal region of the fVIII precursor (“pools 1 to 24”). Both in the studies in human subjects, and in hemophilia mice, the use of fVIII domain pools, immediately followed by screening with the individual peptides, appears to be the most effective strategy. This is likely because a number of epitopes is recognized on each domain: thus, the use of the pools 1 to 24 does not allow to exclude any of them from further investigation of that sequence region for the presence of CD4+ epitopes.

Hemophilia Patients Response to fVIII Peptide Pools. The CD4⁺ response to fVIII in four hemophilia A patients with inhibitors, three hemophilia A patients without inhibitors and four patients with acquired (autoimmune) hemophilia was studied. CD8⁺ depleted, CD4⁺ enriched blood lymphocytes (hereafter referred to as CD4⁺ BL) of these patients was obtained approximately every month, for up to six months. The response of the CD4⁺ BL to increasing concentrations of fVIII and to the individual fVIII domain pools was tested.

The CD4+ response was not constant: in most patients it was detectable at most, but not all, the time points tested. When present, the intensity of the response generally increased with the concentration of fVIII used in the assay. In most cases it reached a maximum at concentrations around I unit of fVIII/mL (i.e., similar to the physiologic concentration of fVIII in the blood in normal subjects).

Although all patients had a significant CD4⁺ response to fVIII in most experiments, several patients had brief periods of time when a CD4⁺ response to fVIII could not be detected. The CD4⁺ BL of most patients in all groups recognized most or all the fVIII domain pools. Like the CD4⁺ response to fVIII, the responses to the domain pools were not stable over time in their intensity: for most patients, the response to one or more of the domain pools decreased for short periods to undetectable levels.

The data indicated that the CD4⁺ BL recognized the different fVIII domain pools with different intensity. Most patients, and all three groups, had very similar patterns of recognition of the fVIII domains. This supports the hypothesis that universal, immunodominant CD4⁺ epitopes exist for fVIII, as they do for the other antigens. Domains A3, A1, or both were the most strongly recognized in all groups and in all patients.

In all patients, the concentration of anti-fVIII antibodies at the time of the experiment testing the response to fVIII of the CD4+ BL was determined. As expected, the correlation between these two parameters was loose.

Proliferative Response over Time in Healthy Subjects. The “danger” theory of tolerance predicts that the immune response does not discriminate on the basis of “self” and “non-self”, but rather whether an Ag is perceived as potentially dangerous or not. Self-proteins processed and presented to the CD4⁺ cells in the context of “danger” situations (i.e., by professional APC at the site of an inflammatory reaction) will become the target of a CD4⁺ cell response. fVIII might be recognized by CD4⁺ cells in healthy controls due to is extravasation at hemorrhagic sites such as bruises, where fVIII sequences may be presented by APC able to prime potentially autoreactive CD4⁺ cells specific for fVIII epitopes. To test this model, monthly, for up to 13 months, the proliferative response to fVIII of blood CD4⁺ cells from 12 healthy subjects was tested.

In all subjects, transient, yet significant and sometimes vigorous responses to fVIII were observed. The activated anti-fVIII CD4+ cells disappear in one or more months, possibly as a result of anergy or deletion by peripheral mechanisms of tolerance, in the presence of the high normal blood levels of fVIII. Such cells would persist in hemophilia A patients because their low fVIII levels, even after periodic replacement therapy, would not suffice for tolerization of the autoreactive CD4⁺ cells. Circumstantial evidence in support of this model is the negative correlation that has been described between development of inhibitors and presence of circulating “fVIII Ag” (Reisner et al., 1995, however, see also McMillan et al., 1988). These findings are consistent with the presence of low levels of Ab to fVIII in normal people (Gilles et al., 1994; Algiman at el., 1992; Batlle et al., 1996). That fVIII may be commonly processed and presented by class II molecules in healthy humans is supported by the finding that a fVIII-derived peptide was eluted from purified human DR molecules (Chicz et al., 1993).

The response of CD4⁺ BL from the same 12 subjects to the fVIII peptide pools 1-24 described above was tested. Two sets of experiments were carried out, roughly one year apart, with the same set of subjects and with overall consistent results. The results obtained in both experiments had a similar overall pattern. All subjects recognized several peptide pools. Pools within the sequence of the A1, A2, A3, C1 and C2 domains were recognized more strongly and more frequently than those spanning the B domain. Anti-fVIII Abs, including the inhibitors in hemophilia patients recognize primarily (but not exclusively) epitopes formed by the A2, A3 and C2 domains (Scanella, 1996; Scanella et al., 1995; Healy et al., 1995; Shima et al., 1995). Thus, these domains may dominate both the pathogenic immune response to fVIII that leads to inhibitor formation in hemophilia A and the ephemeral, non-pathogenic responses of healthy subjects.

The CD4⁺ response of 11 healthy subjects to the fVIII domain pools was over time. Towards this goal the CD4 BL of 11 healthy subjects was challenged every one-three months, up to four times. The CD4+ BL were tested in proliferation assays, using each of the fVIII domain pools. The pattern observed was reminiscent of that observed in the hemophilia A patients, although several subjects had overall low responses to the fVIII domain pools. The responses observed were not stable over time. Positive responses may be followed or preceded by absence of response to the same fVIII domain pools.

The fVIII domain pools A3, C2 and C1 were the most strongly recognized. The domain pools A1 and B1 were the least strongly recognized overall.

### EXAMPLE II

### Initial Studies in Hemophilia A Mice

Mutant mice have been developed with targeted gene disruption of the fVIII gene, that results in severe fVIII deficiency (Bi et al., 1995). These mutant fVIII deficient mice (hereafter referred to as hemophilia A mice) are an excellent model of hemophilia A, including the development of fVIII inhibitor Ab and of a CD4⁺ response after intravenous (i.v.) exposure to human fVIII (Qian et al., 1997; Qian et al., 1996). Hemophilia A mice develop anti-fVIII Ab after two or three i.v. infusions of 0.2 mg of human fVIII (an exposure comparable, on a weight basis, to that given in hemophilia A patients) (Ding et al., 1993; Macatonia et al., 1993). The concentration of serum anti-fVIII Ab increases with the number of exposures to fVIII, and the dose used. All mice that were injected five times with human fVIII have inhibitors (Ding et al., 1993; Macatonia et al., 1993) Approximately 50% of the hemophilia A mice treated with human fVIII i.v. have a detectable proliferative response of spleen T cells (Macatonia et al., 1993).

### EXAMPLE III

### Tolerization of Hemophilia A Mice

To determine whether hemophilia A mice that had encountered human fVIII through intravenous administrations develop a response to fVIII reminiscent of that observed in hemophilia A patients, mice with a targeted gene disruption of exon 17 of the fVIII gene were used (Bi et al., 1995; Bi et al., 1996). These mice have less than 1% of the normal plasma fVIII activity, impaired hemostasis, severe bleeding after minor injuries, subcutaneous and intramuscular bleeding after routine handling, and spontaneous bleedings (Bi et al., 1995; Quian et al., 1999; Bi et al., 1996; Muchitsch et al., 1997).

Mice were treated up to 10 times intravenously with 1 μg of purified recombinant human fVIII. CD8⁺ depleted spleen cells (so as to leave only CD4⁺ T cells; hereafter referred to as “CD4⁺ splenocytes”) were used in proliferation assays to test the response to increasing concentrations of fVIII (5-20 nM) and to pools of overlapping synthetic peptides, spanning the sequence of the individual the fVIII domains (fVIII domain pools). Also, the cytokines secreted by CD4⁺ splenocytes, after challenge in vitro with fVIII, were determined. The anti-fVIII antibody was measured in the sera as well as their IgG subclass (by ELISA). The inhibitors were measured by the Bethesda assay (Kasper et al., 1975).

Results

CD4⁺ splenocytes from mice that received three or more fVIII administrations proliferated consistently and vigorously when exposed to human fVIII, and recognized all fVIII domain pools. Their responses declined after nine or more fVIII administrations, suggesting that frequent administrations of generous doses of fVIII caused immune tolerization. Antibodies to human fVIII and inhibitors appeared in the mouse blood after four to five administrations of fVIII. They were mostly IgG1 (equivalent to human IgG4; Abbas et al., 1997) and to a lesser extent IgG2a (equivalent to human IgG1; Abbas et al., 1997). Thus, like in hemophilia A patients, both Th2 and Th1 cells drive the anti-fVIII antibody synthesis. CD4+ splenocytes from fVIII-treated mice, after challenge in vitro with fVIII, secreted IL-10. In several mice they also secreted IFN-γ, but they never secreted IL-2. Thus, these mice, like hemophilia A patients, mount CD4 and antibody responses after intravenous administration of fVIII. Their anti-fVIII antibody can be inhibitors, belong to IgG subclasses homologous to those of the inhibitors in hemophilic patients, and both the Th2 cytokine, IL-10, and the Th1 cytokine, IFN-γ, may be involved in their synthesis.

Epitope Repertoire on Human fVIII Recognized be CD4⁺ Cells from Hemophilia A Mice. The epitope repertoire of the CD4⁺ cells sensitized to human fVIII in hemophilia A mice was examined. Mice were immunized by multiple subcutaneous injections of 5-10 μg recombinant human fVIII, emulsified in Freund's adjuvant. This ensured a stronger sensitization of the CD4⁺ cells than that obtained after intravenous administration of fVIII. The use of CD4+ splenocytes from mice strongly sensitized to fVIII increases the chances of identifying a comprehensive CD4⁺ repertoire, because the CD4⁺ cells recognizing individual epitopes on an antigen are scarce among unselected CD4⁺ splenocytes.

Four independent groups of 2 to 4 hemophilia A mice were immunized. For each group their CD4⁺ splenocytes were pooled, and tested for their proliferative response to fVIII, and to the individual synthetic peptides spanning the sequence of human fVIII. Because of the large number of peptides, all the peptides were tested only in one experiment. In two experiments, peptides spanning the A and C domains were tested. In a fourth experiment, only peptides spanning the Al domain were tested.

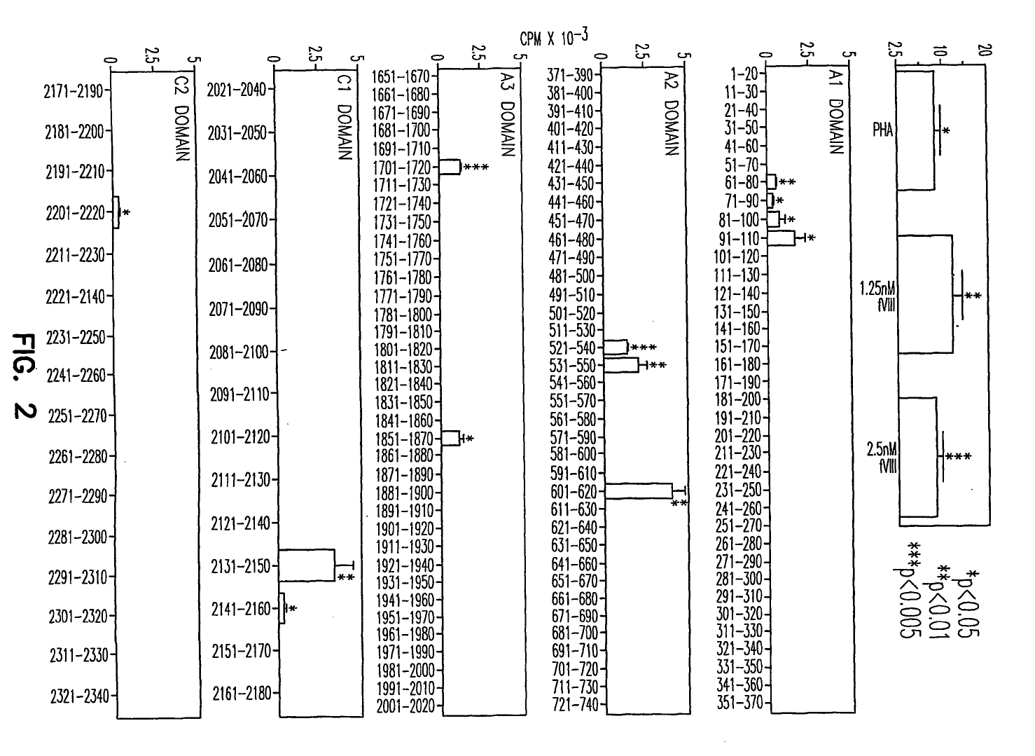

In all experiments the CD4⁺ splenocytes proliferated vigorously in response to human fVIII. In spite of some individual variations in the repertoire of fVIII peptides recognized, a few peptides were recognized clearly and consistently in all or most experiments. They included: on the A1 domain, peptide 71-90 and in some experiments the peptides flanking this sequence; on the A2 domain, peptides 521-550 and 601-620; on the A3 domain, peptide 1701-1720; on the C1 domain, the overlapping peptides 2131-2150 and 2141-2160; and on the C2 domain peptide 2201-2220. FIG. 2 reports the result of a representative experiment.

Peripheral Tolerization with Synthetic Peptide Sequences of fVIII Strongly Suppresses the Mouse's Ability to Synthesize Anti-fVIII Antibodies. Previous studies that used mouse experimental myasthenia gravis (an antibody-mediated autoimmune disease) as a model, demonstrated that nasal or subcutaneous administration of a limited number of short synthetic CD4⁺ epitope sequences of the nicotinic acetylcholine receptor (the target autoantigen in this disease), or even of a single CD4⁺ epitope sequence of the acetylcholine receptor, effectively prevented the development of anti-acetylcholine receptor antibodies when the animals were immunized with this antigen (Karachunski et al., 1997).

As a first step to determine whether peptide-based tolerance induction would be a viable therapeutic measure to prevent formation of inhibitors in hemophilia A, the effect of nasal and intravenous administration of synthetic CD4 epitopes of fVIII to hemophilia A mice was examined. For these experiments a pool of six 20-residue synthetic peptides was used, corresponding to the sequence regions of human fVIII that had been identified as forming epitopes for CD4+ cells. Those sequences were: residues 71-90 of the A1 domain, residues 531-550 and 601-620 of the A2 domain, residues 2131-2150 and 2141-2160 of the C1 domain, and residues 2201-2220 of the C2 domain. This peptide pool was administered to the mice by a trans-nasal route, as described in Karachunski et al. (1997). A group of 8 mice was treated with the peptides. The mice received 50 μg of each peptide twice a week for three weeks before the beginning of the intravenous administrations of human fVIII. A second group of 7 control mice was treated nasally with PBS only. After the beginning of the treatment with fVIII, the peptides (or PBS only) were administered nasally only once per week. Each mouse received 1 μg of fVIII intravenously every two weeks for a total of up to nine injections. The mice that had been treated nasally with the fVIII peptides received intravenous injections of fVIII mixed with the epitope peptide pool (251 g of each peptide in each injection). The control mice sham treated with clean PBS received intravenous administrations of fVIII without any peptide.

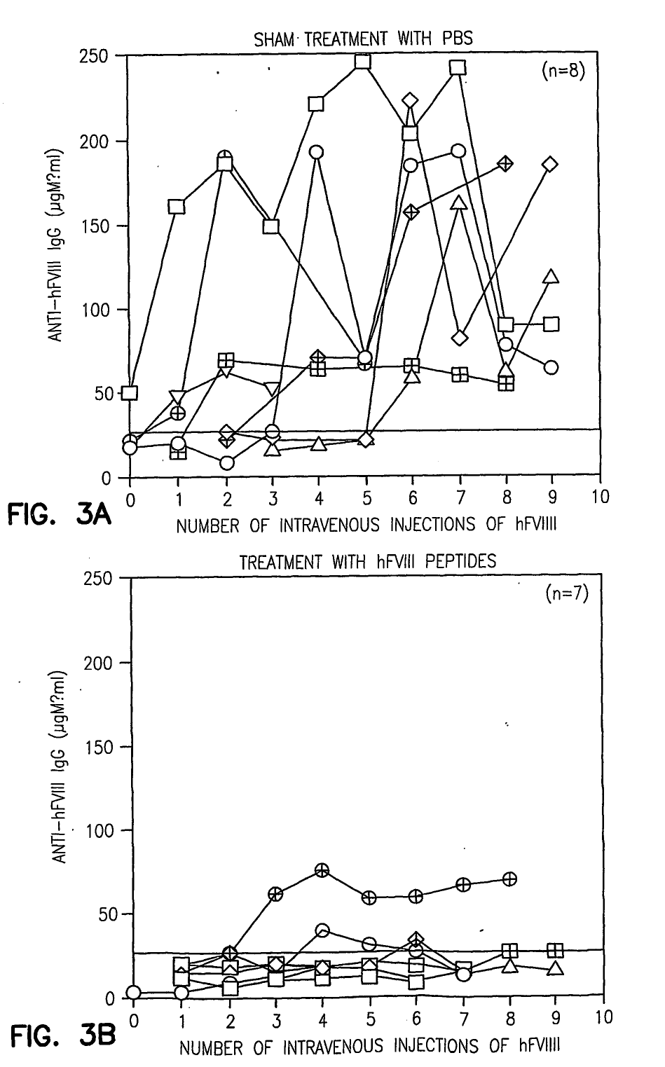

Blood was obtained from the mice two weeks after each intravenous injection of fVIII. In some mice blood was also obtained just before the beginning of the fVIII treatment. Blood was not obtained from every mouse after every fVIII treatment, because of the propensity of hemophilic mice to bleed and the difficulties in obtaining blood from the tail vein. The concentration of anti-human fVIII IgG in the mouse sera was measured by ELISA. The results of this assay are expressed as μg/mL of fVIII-specific IgG. Sera from mice that had not received any treatment with fVIII or with fVIII sequences yielded values lower than 25 μg/mL. Thus, anti-fVIII IgG values lower than 25 μg/mL are considered as background. FIG. 3 reports the results obtained in the peptide treated mice (right panel) and in the mice sham-tolerized with PBS alone (left panel). The shaded areas at the bottom of each graph include the values lower than 25 μg/mL, which should be considered as background. The mice treated with fVIII and sham tolerized with clean PBS clearly produced anti-fVIII IgG antibodies. On the other hand, only one mouse tolerized with fVIII peptides developed consistent, albeit modest, anti-fVIII antibodies. Another two peptide-tolerized mice developed transient, minimal amounts of anti-fVIII antibodies.

### EXAMPLE IV

### Regions of fVIII That Form Universal Immunodominant Epitopes for Sensitization of CD4⁺ Cells in Humans

In order to develop therapeutic approaches for induction of immune tolerance to fVIII based on the use of CD4⁺ epitope sequences, the sequence of the regions of fVIII recognized by CD4⁺ cells in hemophilia patients with inhibitors, and in humans in general needs to be identified. In addition, to develop practical tolerance procedures, immunodominant, universal epitopes recognized by fVIII-specific CD4⁺ cells in most if not all humans need to be identified so that the individual epitope repertoire recognized by the fVIII-specific CD4⁺ cells in each patient would not be needed. Moreover, these identified sequences may be used to prevent the appearance of inhibitors in infants, before their first therapeutic exposure to fVIII.

As described below, immunodominant, universal CD4⁺ epitopes on two of the three domains of human fVIII that are recognized by antibody inhibitors were identified. Specifically, the sequence regions of the A3 and C2 domains that form epitopes recognized by CD4⁺ cells were determined in several groups of hemophilia A patients, acquired hemophilia patients and in healthy subjects. It should be noted that, as expected from the frequent presence in healthy blood donors of low titers of anti-fVIII IgG antibodies, healthy subjects frequently have CD4⁺ cells sensitized to fVIII, although the intensity and the frequency of CD4⁺ responses to fVIII in healthy subjects are lower than those of the anti-fVIII CD4⁺ responses we observed in hemophilia A patients (Reding et al., 2000).

Materials and Methods.

Peptide Selection and Synthesis. A library of overlapping peptides was synthesized according tot he method of Houghten (1985), spanning the amino acid sequence of the fVIII A3 and C2 domains (Vehar et al., 1984) (Tables 2 and 3). The A3 and the C2 domains contain binding sites for antibody inhibitors.

These domains are of particular interest because the crystal structure of the C2 domain allows for correlation of the structural features of the CD4⁺ epitopes with their immune dominance and the A3 domain is strongly recognized by most hemophilia patients and healthy subjects, including hemophilia A and acquired hemophilia (Reding et al., 2000). It was found that 71% of the hemophilia A patients with inhibitors, 94% of the hemophilia A patients without inhibitors, 79% of acquired hemophilia patients, and 70% of healthy subjects have CD4⁺ cells that recognize the A3 domain (Reding et al. 2000). Thus, the A3 domain is an ideal candidate for forming immunodominant, universal epitopes. Although its crystal structure has not been solved, the A3 domain is highly homologous to the A domains of ceruloplasmin, whose crystal structure is known (Zaitseva et al., 1996) and can be used to construct models of the A3 domain structure (Pemberton et al., 1997).

The peptides were 20 residues long (apart from the carboxyl terminal peptide of C2, which was 13 residues), and their sequences overlapped by 10 residues. Their length compares with that of naturally processed class II restricted epitopes, which are 9-14 residues (Stern et al., 1994). The sequence overlap reduces the risk of missing epitopes “broken” between peptides. A solution of either the individual peptides, or of roughly equimolar pools of all the peptides spanning the sequence of the A3 or the C2 domains (A3 and C2 domain pools) were used.

The peptides synthesized by this method are 70-85% pure (Houghten, 1985; Protti et al., 1990; Manfredi et al., 1992). Contaminants are a mixture of shorter analogs in which one or more residues are missing randomly, due to incomplete coupling. The analogs might bind the class II molecule, but not the specific T cell receptor in a manner conducive to measurable T cell response. This may cause a shift of the dose dependence of the CD4⁺ cell responses to the peptide, towards higher doses than when using purified peptides. Because the doses used to test anti-fVIII CD4⁺ cells were generous, the risk of missing detection of the response to a peptide because of the presence of contaminating analogs is very small.

The sequence and purity of several peptides, selected randomly, was checked by determination of their amino acid composition (Heinrickson et al., 1983) and the molecular weight of the species present in the peptide preparation by mass-spectrometry. Amino acid composition analysis yielded results corresponding to the theoretical values for all peptides. Mass-spec analysis consistently yielded a major peak with the molecular weight calculated for the peptide.

Subiects. Six HIV negative severe hemophilia A patients (3 with inhibitors and 3 without), 4 acquired hemophilia patients (Table 4) and 6 healthy subjects (3 men and 3 women, 34-48 years old) were studied. The acquired hemophilia patients had not received fVIII during the period of time when we studied their CD4⁺ response to the fVIII peptides. Among hemophilia A patients, patient #8 had received immune tolerance therapy with high doses of fVIII. The last high dose of fVIII administered as part of the immune tolerance therapy was given approximately 8 months before the first experiment reported here. The therapy was not successful since the inhibitor persisted, with titers that frequently were comparable to those observed just before beginning the immune tolerance therapy (Reding et al., 2000). Patient #9 received a prophylactic regimen of weekly injections of standard therapeutic doses of fVIII. All other patients were self treated on an as needed basis. In several hemophilic and healthy subjects we tested the response of CD4⁺ blood lymphocytes to fVIII peptides on more than one occasion, at intervals that ranged from a few weeks to several months.

Preparation of CD4⁺ Blood Lymphocytes and Proliferation Assay.

Peripheral blood mononuclear cells (PBMC) were isolated from venous blood and depleted them of CD8⁺ T cells (Manfredi et al., 1993), using anti-human CD8 antibody (OKT8; Ortho Diagnostic Systems, Raritan, N.J. or Ancell, Bayport, Minn.) and paramagnetic beads coated with goat anti-mouse IgG antibody (PerSeptive Biosystems, Framingham, Mass.). The CD8⁺ depleted PBMC (CD4⁺ blood lymphocytes) were used for 5 day proliferation assays (Manfredi et al., 2993), using sextuplet wells (2×10⁵ cells/well; when cell yield was low, a minimum of 1×10⁵ cells/well) and the following stimulants: phytohemagglutinin (Sigma; 10 μg/mL), T cell growth factor (Lymphocult; Biotest Diagnostic Corp., Danville, NJ; final concentration of interleukin 2, 10 U/mL), recombinant human fVIII (Baxter, Glendale, Calif. or Bayer, Elkhart, Ind.; 0.5-1 units/mL; normal plasma concentration: I unit/mL), and the synthetic fVIII peptides, both individually and in pools as described above (final concentration 2 μg/mL of each peptide). Sextuplet wells cultured without any stimulus provided the basal proliferation rate of the cells. We measured cell proliferation from the incorporation of ³H-thymidine (1 μCi per well, specific activity 6.7 Ci/mmol; Dupont-NEN, Boston, Mass.), expressed as counts per minute (cpm). When an antigen induced a statistically significant (p<0.05) increase in proliferation (assessed using a two-tailed Student's t test), we calculated the stimulation index (SI: ratio between average cpm of cultures in the presence of the antigen and average basal proliferation of the same cells). The use of SI normalizes results, and allows comparison of experiments carried out at different times, and with different subjects.

TABLE 2
A3 peptides.

| Position of the first | |
|---|---|
| and last peptide | |
| residue on the fVIII | |
| precursor sequence | Peptide sequence |
| 1651-1670 | ITRTTLQSDQEEIDYDDTIS |
| | (SEQ ID NO: 53) |
| 1661-1680 | EEIDYDDTISVEMKKEDFDI |
| | (SEQ ID NO: 54) |
| 1671-1690 | VEMKKEDFDIYDEDENQSPR |
| | (SEQ ID NO: 55) |
| 1681-1700 | YDEDENQSPRSFQKKTRHYF |
| | (SEQ ID NO: 56) |
| 1691-1710 | SFQKKTRHYFIAAVERLWDY |
| | (SEQ ID NO: 57) |
| 1701-1720 | IAAVERLWDYGMSSSPHVLR |
| | (SEQ ID NO: 58) |
| 1711-1730 | GMSSSPHVLRNRAQSGSVPQ |
| | (SEQ ID NO: 59) |
| 1721-1740 | NRAQSGSVPQFKKVVFQEFT |
| | (SEQ ID NO: 60) |
| 1731-1750 | FKKVVFQEFTDGSFTQPLYR |
| | (SEQ ID NO: 9) |
| 1741-1760 | DGSFTQPLYRGELNEHLGLL |
| | (SEQ ID NO: 10) |
| 1751-1770 | GELNEHLGLLGPYIRAEVED |
| | (SEQ ID NO: 11) |
| 1761-1780 | GPYIRAEVEDNIMVTFRNQA |
| | (SEQ ID NO: 12) |
| 1771-1790 | NIMVTFRNQASRPYSFYSSL |
| | (SEQ ID NO: 13) |
| 1781-1800 | SRPYSFYSSLISYEEDQRQG |
| | (SEQ ID NO: 14) |
| 1791-1810 | ISYEEDQRQGAEPRKNFVKP |
| | (SEQ ID NO: 15) |
| 1801-1820 | AEPRKNFVKPNETKTYFWKV |
| | (SEQ ID NO: 16) |
| 1811-1830 | NETKTYFWKVQHHMAPTKDE |
| | (SEQ ID NO: 17) |
| 1821-1840 | QHHMAPTKDEFDCKAWAYFS |
| | (SEQ ID NO: 18) |
| 1831-1850 | FDCKAWAYFSDVDLEKDVHS |
| | (SEQ ID NO: 19) |
| 1841-1860 | DVDLEKDVHSGLIGPLLVCH |
| | (SEQ ID NO: 20) |
| 1851-1870 | GLIGPLLVCHTNTLNPAHGR |
| | (SEQ ID NO: 21) |
| 1861-1880 | TNTLNPAHGRQVTVQEFALF |
| | (SEQ ID NO: 22) |
| 1871-1890 | QVTVQEFALFFTIFDETKSW |
| | (SEQ ID NO: 23) |
| 1881-1900 | FTIFDETKSWYFTENMERNC |
| | (SEQ ID NO: 24) |
| 1891-1910 | YFTENMERNCRAPCNIQMED |
| | (SEQ ID NO: 25) |
| 1901-1920 | RAPCNIQMEDPTFKENYRFH |
| | (SEQ ID NO: 26) |
| 1911-1930 | PTFKENYRFHAINGYIMDTL |
| | (SEQ ID NO: 27) |
| 1921-1940 | AINGYIMDTLPGLVMAQDQR |
| | (SEQ ID NO: 28) |
| 1931-1950 | PGLVMAQDQRIRWYLLSMGS |
| | (SEQ ID NO: 29) |
| 1941-1960 | IRWYLLSMGSNENIHSIHFS |
| | (SEQ ID NO: 30) |
| 1951-1970 | NENIHSIHFSGHVFTVRKKE |
| | (SEQ ID NO: 31) |
| 1961-1980 | GHVFTVRKKEEYKMALYNLY |
| | (SEQ ID NO: 32) |
| 1971-1990 | EYKMALYNLYPGVFETVEML |
| | (SEQ ID NO: 33) |
| 1981-2000 | PGVFETVEMLPSKAGIWRVE |
| | (SEQ ID NO: 34) |
| 1991-2010 | PSKAGIWRVECLIGEHLHAG |
| | (SEQ ID NO: 35) |
| 2001-2020 | CLIGEHLHAGMSTLFLVYSN |
| | (SEQ ID NO: 36) |

TABLE 3
C2 peptides.

| Position of the first | |
|---|---|
| and last peptide | |
| residue on the fVIII | |
| precursor sequence | Peptide sequence |
| 2161-2180 | STLRMELMGCDLNSCSMPLG |
| | (SEQ ID NO: 61) |
| 2171-2190 | DLNSCSMPLGMESKAISDAQ |
| | (SEQ ID NO: 37) |
| 2181-2200 | MESKAISDAQITASSYFTNM |
| | (SEQ ID NO: 38) |
| 2191-2210 | ITASSYFTNMFATWSPSKAR |
| | (SEQ ID NO: 39) |
| 2201-2220 | FATWSPSKARLHLQGRSNAW |
| | (SEQ ID NO: 40) |
| 2211-2230 | LHLQGRSNAWRPQVNNPKEW |
| | (SEQ ID NO: 41) |
| 2221-2240 | RPQVNNPKEWLQVDFQKTMK |
| | (SEQ ID NO: 42) |
| 2231-2250 | LQVDFQKTMKVTGVTTQGVK |
| | (SEQ ID NO: 43) |
| 2241-2260 | VTGVTTQGVKSLLTSMYVKE |
| | (SEQ ID NO: 44) |
| 2251-2270 | SLLTSMYVKEFLISSSQDGH |
| | (SEQ ID NO: 45) |
| 2261-2280 | FLISSSQDGHQWTLFFQNGK |
| | (SEQ ID NO: 46) |
| 2271-2290 | QWTLFFQNGKVKVFQGNQDS |
| | (SEQ ID NO: 47) |
| 2281-2300 | VKVFQGNQDSFTPVVNSLDP |
| | (SEQ ID NO: 48) |
| 2291-2310 | FTPVVNSLDPPLLTRYLRIH |
| | (SEQ ID NO: 49) |
| 2301-2320 | PLLTRYLRIHPQSWVHQIAL |
| | (SEQ ID NO: 50) |
| 2311-2330 | PQSWVHQIALRMEVLGCEAQ |
| | (SEQ ID NO: 51) |
| 2321-2332 | RMEVLGCEAQDLY |
| | (SEQ ID NO: 52) |

TABLE 4
Acquired Hemophilia Patients

| Patient | Age | Sex | Inhibitor Status¹ | Maximum InhibitorTiter² |
|---|---|---|---|---|
| 1 | 39 | F | − | low |
| 2 | 58 | M | − | high |
| 3 | 63 | M | − | not tested |
| 4 | 74 | M | + | high |
| Patient | Age | Sex | Inhibitor Status¹ | Maximum InhibitorTiter³ |

Hemophilia A Patients with Inhibitors

| | | | | |
|---|---|---|---|---|
| 5 | 43 | M | + | 2 |
| 6 | 35 | M | not tested | 2100 |
| 8 | 26 | M | + | 4833 |

Hemophilia A Patients without Inhibitors

| | | | | |
|---|---|---|---|---|
| 9 | 53 | M | N/A | N/A |
| 10 | 44 | M | N/A | N/A |
| 12 | 24 | M | N/A | N/A |

¹At the time of experiments. (+) inhibitor present, (−) inhibitor not present
²Based on PTT inhibitor inactivator assay. Inhibitors seen at 1:4 or smaller dilutions are indicated as “low titer”; inhibitors seen at 1:16 or greater dilutions are indicated as “high titer”.
³Bethesda units/mL

Results

Response of Blood CD4⁺ Lymphocytes to A3 Peptides. The proliferative response of CD4+ lymphocytes from 6 healthy subjects and the hemophilia patients (Table 4) to individual peptides spanning the A3 domain was tested. Most subjects were studied on two or more occasions. The different experiments testing the response of the same subjects were done from a few weeks to several months apart. All subjects responded to one or more peptides in at least one experiment, with the exception of patient #10 who did not respond to any A3 peptide in either of the two experiments carried out with his CD4+ cells. The occasional lack of response to A3 peptides in subjects who responded strongly at other times is consistent with the intermittent nature of the CD4 response to fVIII and fVIII domain pools that have been described (Reding et al., 2000).

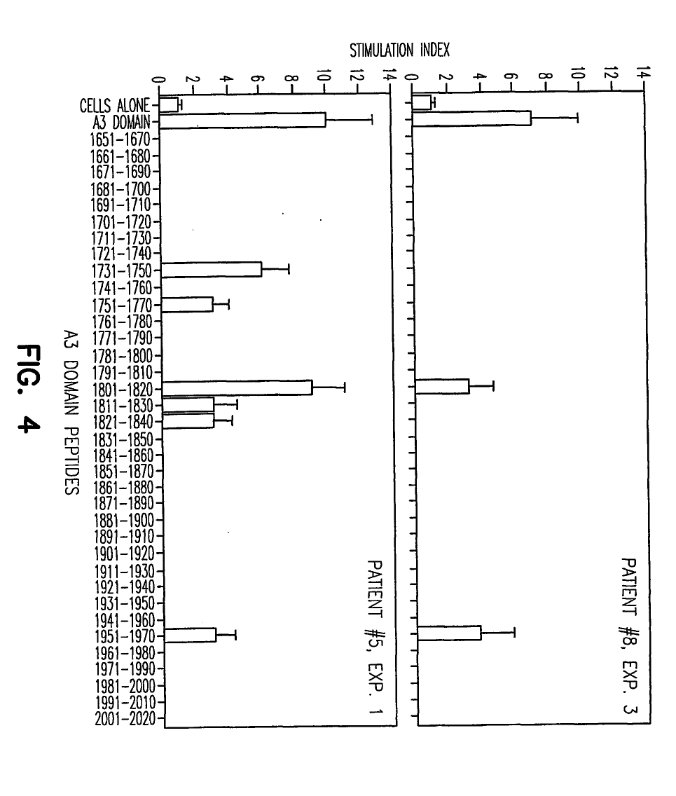

Most subjects recognized several A3 peptides. FIG. 4 shows the results obtained in experiments carried out with the CD4+ cells from two hemophilia A patients with inhibitors: the intensity of the responses and the scattering of the data is representative of those obtained in all experiments in which a significant response to individual peptides spanning the sequence of the A3 or C2 domains was found. The peptides that elicited the strongest proliferative response varied in the different subject groups (Table 5).

Peptide 1691-1710 was strongly recognized in 7 of the 8 experiments done with CD4⁺ cells from healthy subjects: healthy subject #8 recognized strongly in the second experiment the two peptides, 1681-1700 and 1701-1720 that overlap the sequence of peptide 1691-1710 at its amino terminal and carboxyl terminal ends. Healthy subject #2 did not recognize peptide 1691-1710. However, healthy subject #2 recognized the sequence region immediately after its carboxyl terminal residue (residues 1711-1730). The peptides comprising the sequence region 1681-1720 were recognized in 6 of 19 experiments with CD4⁺ cells from hemophilia patients. Peptide 1691-1710 was strongly recognized in 3 hemophilia A patients (2 with inhibitors, 1 without), and in 1 acquired hemophilia patient, but in only one of the different experiments carried out with CD4⁺ cells of the same patients. Among hemophilia A patients, patient #8 recognized the sequence 1691-1710 in one experiment. The overlapping sequence 1701-1720 (patients #2 and #8) and 1671-1690 (patient #8) was recognized in another experinent. Also the sequence region 1941-1980 was recognized frequently by the CD4⁺ cells from healthy subjects, but less frequently by the CD4⁺ cells from hemophilia patients, irrespective of their inhibitor status: one or more peptides spanning this sequence were recognized in 5 of 8 experiments done with CD4⁺ cells from healthy subjects, but in only 2 of 19 experiments done with CD4⁺ cells from hemophilia patients.

The recognition of peptides spanning the sequence 1791-1840 correlated with the presence of inhibitors. Peptides in this sequence region were recognized in 6 of the 7 experiments done with CD4⁺ cells from hemophilia A patients with inhibitors, but in only 3 of 8 experiments done with CD4⁺ cells from healthy subjects, and in none of the experiments using CD4⁺ cells from hemophilia A patients without inhibitors. This region was recognized by all the 3 acquired hemophilia patients studied, although its recognition was not consistent in the different experiments that tested the same patient. Usually peptides within this sequence segment were strongly recognized by CD4⁺ cells from acquired hemophilia patients when their CD4⁺ cells displayed a substantial reactivity to A3 peptides. Patients #1 and #2 also recognized in some experiments peptide 1831-1859 which overlaps with 1791-1840.

Response of Blood CD4⁺ Lymphocytes to C2 Peptides, The proliferative response to individual C2 domain peptides of CD4⁺ lymphocytes from 3 healthy subjects and 9 hemophilia patients was tested (Table 6). Similar to studies on the CD4⁺ response to the A3 peptides, some subjects were studied on two or more occasions. All subjects responded to one or more peptides in at least one experiment, with the exception of healthy subject #8 and patient #4. As seen with the A3 peptides, the occasional lack of response to C2 peptides in subjects who responded strongly at other times is consistent with the intermittent nature of the CD4⁺ response to fVIII and fVIII domain pools that have been described (Reding et al., 2000).

Similar to the A3 peptides, most subjects recognized several of the C2 peptides. In contrast to the A3 peptides, the pattern of C2 peptides that elicited the strongest proliferative response was similar in healthy subjects and in each of the hemophilia patient groups (Table 6). When there was a significant proliferative response to the C2 peptides, the peptides spanning the sequence regions 2181-2240 and/or 2291-2330 were recognized strongly in all experiments in each subject group. One hemophilia A patient with inhibitors (patient # 8) recognized peptide 2171-2190 which overlaps with 2181-2240 which is recognized by most of the other patients.

TABLE 5
A3 peptides that elicited the strongest response of CD4⁺ lymphocytes
from healthy subjects and hemophilia patients.
Healthy Subjects

| | |
|---|---|
| Subject 1, t = 1 | 1691-1710, 1761-1780, 1941-1960 |
| Subject 1, t = 2 | 1691-1710, 1761-1780, 1821-1840 |
| Subject 2, t = 1 | 1711-1730, 1781-1800, 1941-1960 |
| Subject 3, t = 1 | 1691-1710, 1941-1960, 1951-1970 |
| Subject 8, t = 1 | 1691-1710, 1761-1780, 1801-1820 |
| Subject 8, t = 2 | 1681-1700, 1691-1710, 1701-1720 |
| Subject 9, t = 1 | 1691-1710, 1801-1820, 1941-1960 |
| Subject 10, t = 1 | 1691-1710, 1951-1970, 1961-1980 |

Acquired Hemophilia

| | |
|---|---|
| Patient 1, t = 1 | 1691-1710, 1801-1820, 1831-1850 |
| Patient 1, t = 2 | 1831-1850 |
| Patient 1, t = 3 | no response |
| Patient 2, t = 1 | 1701-1720, 1771-1790, 1831-1850, 1901-1920 |
| Patient 2, t = 2 | 1741-1760, 1941-1960 |
| Patient 2, t = 3 | 1801-1830 |
| Patient 3, t = 1 | 1791-1830 |
| Patient 3, t = 2 | no response |

Hemophilia A with Inhibitors

| | |
|---|---|
| Patient 5, t = 1 | 1731-1750, 1801-1820 |
| Patient 5, t = 2 | 1761-1780, 1801-1820, 1981-2000 |
| Patient 6, t = 1 | 1691-1710, 1801-1820 |
| Patient 6, t = 2 | 1751-1770, 1801-1830, 1981-2000 |
| Patient 8, t = 1 | 1671-1690, 1701-1720, 1731-1750, 1771-1790 |
| Patient 8, t = 2 | 1691-1710, 1731-1750, 1801-1840 |
| Patient 8, t = 3 | 1801-1820, 1951-1970 |

Hemophilia A without Inhibitors

| | |
|---|---|
| Patient 9, t = 1 | 2001-2020 |
| Patient 9, t = 2 | 1691-1710, 1751-1770, 1881-1900 |
| Patient 10, t = 1 | no response |
| Patient 10, t = 2 | no response |

TABLE 6
C2 peptides that elicited the strongest response of CD4⁺ lymphocytes
from healthy subjects and hemophilia patients.
Healthy Subjects

| | |
|---|---|
| Subject 3, t = 1 | 2191-2240, 2251-2270, 2271-2290, 2291-2310 |
| Subject 8, t = 1 | no response |
| Subject 9, t = 1 | 2291-2310 |

Acquired Hemophilia

| | |
|---|---|
| Patient 1, t = 1 | 2191-2210, 2251-2270, 2271-2290, 2301-2330 |
| Patient 1, t = 2 | no response |
| Patient 2, t = 1 | 2191-2210, 2271-2290, 2301-2320 |
| Patient 2, t = 2 | no response |
| Patient 4, t = 1 | no response |

Hemophilia A with Inhibitors

| | |
|---|---|
| Patient 5, t = 1 | 2301-2320, 2311-2330 |
| Patient 5, t = 2 | no response |
| Patient 6, t = 1 | 2181-2200 |
| Patient 6, t = 2 | no response |
| Patient 8, t = 1 | 2171-2190, 2311-2330 |

Hemophilia A without Inhibitors

| | |
|---|---|
| Patient 9, t = 1 | 2191-2240, 2241-2260, 2251-2270, 2281-2300, |
| | 2301-2330 |
| Patient 9, t = 2 | 2211-2230 |
| Patient 9, t = 3 | 2191-2210, 2301-2330 |
| Patient 10, t = 1 | 2201-2230 |
| Patient 12, t = 1 | 2261-2280, 2301-2320 |

The Sequence Regions of the A3 and C2 Domains That Are Frequently Recognized by Human CD4⁺ Cells Have Structural Properties Characteristic of Universal CD4⁺ Epitopes. Some of the structural features that are common to the universal immunodominant epitopes for human CD4⁺ cells on protein antigens appear to be related to structural properties that permit easy proteolytic cleavage, e.g., the universal CD4⁺ epitope sequences in TTX and DTX tend to be flanked by flexible, exposed sequence loops, which would likely be easy targets for proteases. They should allow the universal CD4⁺ epitopes to be easily released from the antigen during its processing.

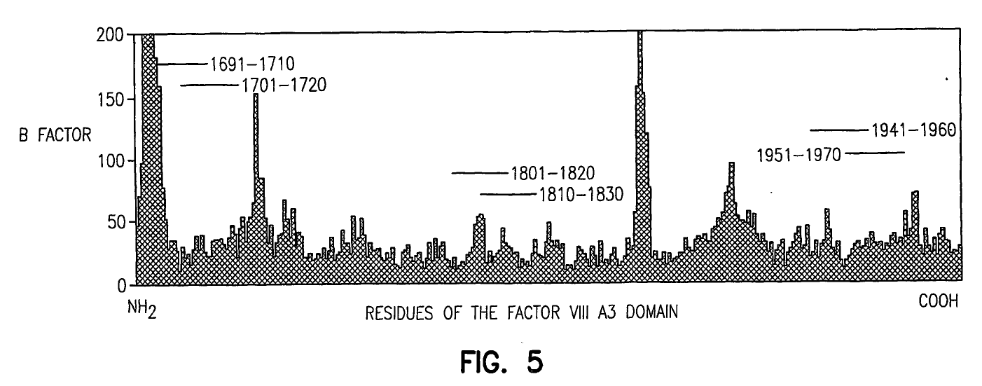

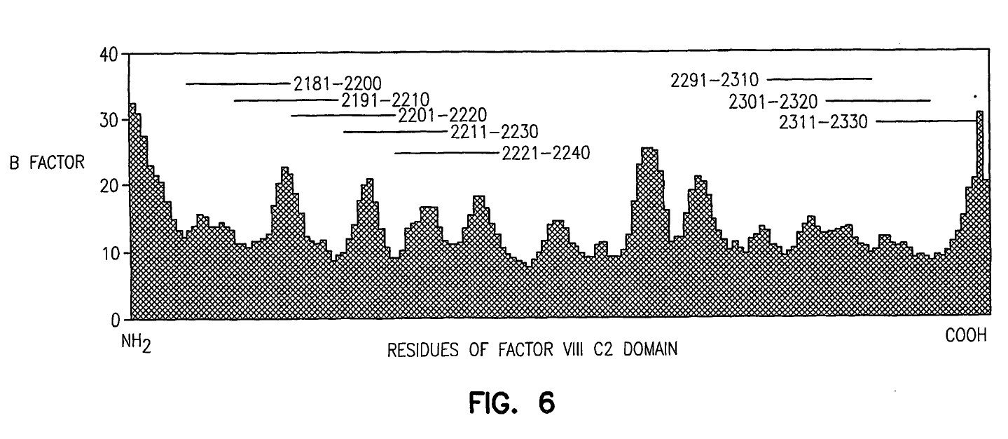

The mobility of a CD4⁺ epitope within a protein antigen, and thus its localized protease sensitivity and subsequent immunodominance of the sequence fragments released most easily, can be predicted by analysis of crystallographic B factors. High B factors correspond to weaker electron density, which is usually the result of movements within the crystal protein lattice. The sequence location of the fVIII peptides identified herein as forming universal CD4⁺ epitopes was compared with the crystallographic B factors of the C2 domain, and of a homology model of the A3 domain based on the known crystal structure of ceruloplasmin. The analysis was limited to the B factor of the α carbons, as they should best reflect the mobility of the peptide backbone. FIGS. 5 and 6 report the results of those analyses.

On the A3 domain (FIG. 5) the sequence regions 1691-1720 and 1941-1970, which are recognized with high frequency by the CD4⁺ cells of healthy subjects, and with some frequency also by the CD4⁺ cells of hemophilia patients, aligned well with valleys in the B factor values, and were flanked at their ends by peaks in the B factors, that indicate a higher mobility of the peptide backbone. The sequence region 1801-1830, which was recognized with high frequency by hemophilia A patients with inhibitors and by acquired hemophilia patients, which also have inhibitors, and with lesser frequency by healthy subjects, comprised two valleys in the B factor values. In the case of the C2 domain (FIG. 6), sequence segments with high B factors were included in and/or flanked the peptides recognized with high frequency by the CD4⁺ cells of all subjects (peptides in the sequence regions 2181-2230 and 2291-2330).

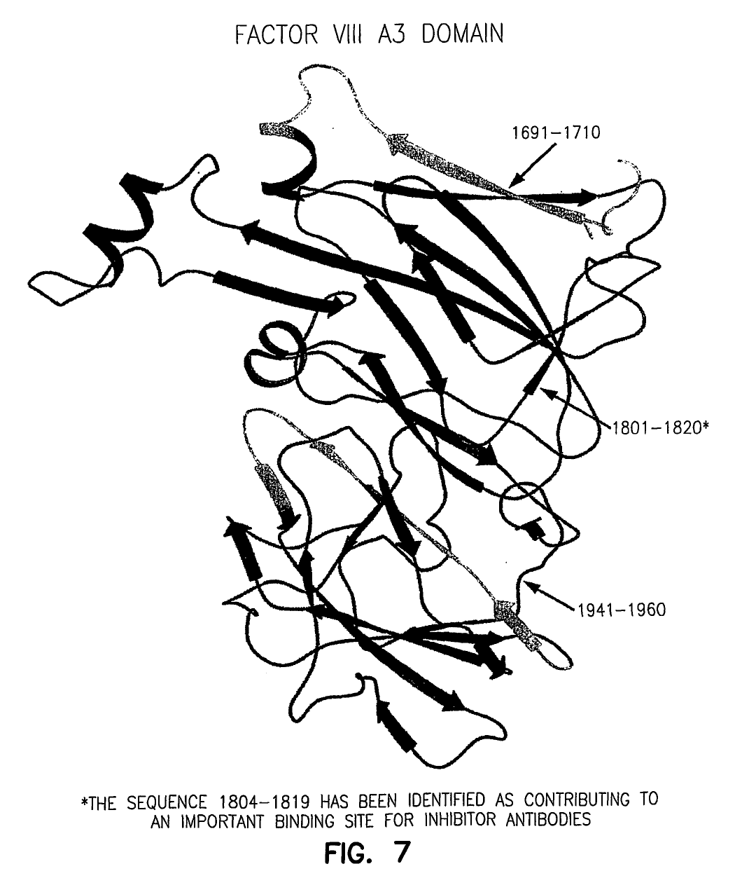

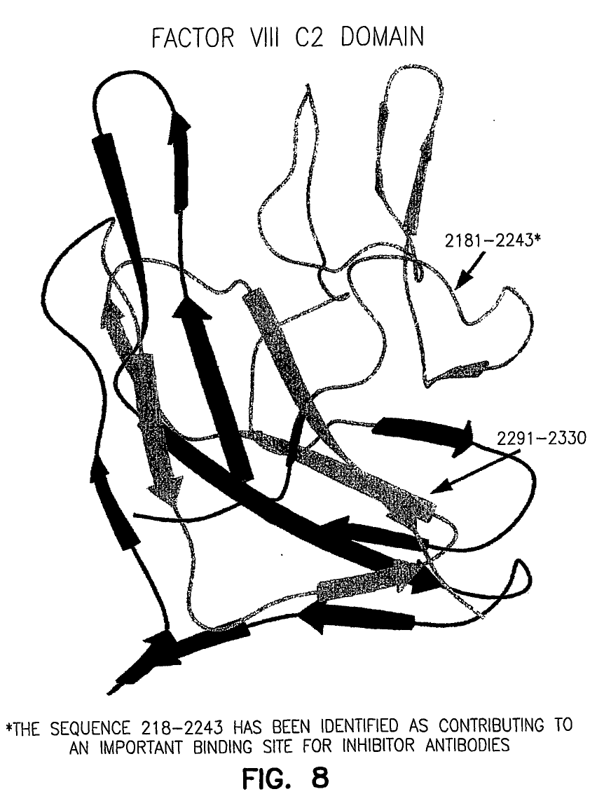

FIG. 7 shows the location of the sequence regions 1691-1710, 1801-1820, and 1941-1960 within the three dimensional structural model of the A3 domain based on the known crystal structure of ceruloplasmin. FIG. 8 shows the location of sequence regions 2181-2240 and 2291-2332 within the three dimensional structure of the C2 domain. These figures demonstrate that significant portions of each of these sequence regions are indeed located in parts of the fVIII molecule that have, or are expected to have a high degree of solvent exposure, thus rendering them easy targets for proteases involved in antigen processing. Also, these models of the three dimensional folding of the A3 and C2 sequences illustrate the presence of relatively unstructured sequence loops in each of the sequence regions that we have identified as forming immunodominant CD4 epitopes. All these structural features are characteristic of universal immunodominant CD4⁺ epitopes.

The CD4+ Epitope Sequence 1801-1820 of the A3 Domain and the CD4⁺ Epitope Sequence 2181-2240 of the C2 Domain Overlap with Sequence Regions of fVIII That Contribute to the Formation of Inhibitor Binding Sites. Several studies have investigated the topographic relationship between the sequence regions that form epitopes for antibodies, and those that are recognized by the CD4⁺ cells that preferentially help B cells that synthesize those antibodies.

The epitopes recognized by antibodies and by CD4⁺ cells are profoundly different. Antibodies recognize three dimensional features on the surface of the antigen molecule. Antibody epitopes are usually made up by residues that are contained in discontinuous sequence regions of the antigen. Those residues are brought into topographic proximity by the three dimensional folding of the protein antigen. Thus, procedures that affect the three dimensional folding of the antigen (i.e., denaturating procedures) will break up the antibody epitopes. On the other hand, CD4⁺ cells recognize linear, denatured peptide fragments of the antigen, associated with class II MHC molecules. Thus, the CD4⁺ T cells and B cells that work together for the synthesis of a given antibody recognize epitopes formed by different residues.

However, there appears to be a preferential cooperation between CD4⁺ and B cells that recognize epitopes between the same domains within a protein antigen. Furthermore, several studies have demonstrated that, albeit structurally different, the epitope sequences recognized by CD4⁺ cells that preferentially cooperate with a given B cell may be very close to the sequence regions that form the antibody epitope. Sometimes, some of the residues that form the CD4⁺ epitope are “inscribed” within the residues that form the antibody epitope (Bellone et al., 1995).

Table I lists the sequence regions of fVIII that have been proposed as contributing residues to the epitopes recognized by inhibitors. Two of the peptide sequences of fVIII that were identified as forming universal CD4⁺ epitopes (1801-1820 in the A3 domain and 2181-2240 in the C2 domain) overlap sequences that likely contribute residues to inhibitor binding sites. This lends further support to the conclusion that the sequence regions described herein contain universal immunodominant CD4⁺ epitopes recognized by CD4⁺ T cells that are involved in the control of inhibitor synthesis. In addition, since residues 2174 and 2326 within the C2 domain are disulfide bonded (McMullen et al., 1995), the amino and carboxyl terminal sequences of C2 are likely in close proximity and may form a single inhibitor binding epitope (Lollar, 1999). This supports the possibility that the universal immunodominant CD4⁺ epitope sequence 2291-2332, which is located at the carboxyl terminal end of the C2 domain, may overlap a sequence segment that contributes residues to a inhibitor binding site.

Thus, in addition to the structural features described above, the overlap with a sequence segment that contributes residues to an inhibitor binding site appears to be predictive of the presence of an immunodominant, universal CD4⁺ epitope on the fVIII sequence.

Prediction of Universal CD4+ Epitope Sequences on the A2 Domain of Human fVIII. The A2 domain, which is homologous to the A3 domain and is likely to fold in a similar manner, also contains binding sites for antibody inhibitors. Because of their homology, the sequences A2 and the A3 domains can be aligned (FIG. 9). The identification of the sequences of the A2 domain that correspond to those identified here as forming immunodominant, universal CD4⁺ epitopes in the A3 domain. Those regions, indicated in color in FIG. 9, correspond to the sequence segments 380-405, 480-535 and 635-671 (the residues are indicated, as throughout this application, according to their position along the sequence of the fVIII precursor). These segments of the A2 sequence likely contain universal CD4⁺ epitopes, and are suitable for induction of tolerance.

Residues within the sequence region 484-508 of the A2 domain are believed to contribute to the formation of an epitope recognized by antibody inhibitors (Lollar, 1999). This sequence region is included in the sequence 480-535 that likely forms universal CD4⁺ epitopes. Thus, the overlap of a sequence segment that contributes to an inhibitor binding site appears predictive of universal CD4⁺ epitopes on the fVIII sequence.

TABLE 7
Sequence Regions of fVIII Predicted or Shown to form Universal,
Immunodominant CD4⁺ Epitopes in Humans.

| | |
|---|---|
| Residues | Sequence |

A2 DOMAIN

| | |
|---|---|
| 380-405 | KTWVHYIAAEEEDWDYAPLVLAPDDR |
| | (SEQ ID NO: 1) |
| 480-535 | ITDVRPLYSRRLPKGVKHLKDFPILPGEIFKY |
| | KWTVTVEDGPTKSDPRCLTRYYSS |
| | (SEQ ID NO: 2) |
| 635-671 | AYWYILSIGAQTDFLSVFFSGYTFKHKMVYE |
| | DTLTLF |
| | (SEQ ID NO: 3) |

A3 DOMAIN

| | |
|---|---|
| 1671-1730 | VEMKKEDFDIYDEDENQSPRSFQKKTRHYFI |
| | AAVERLWDYGMSSSPHVLRNRAQSGSVPQ |
| | (SEQ ID NO: 4) |
| 1791-1850 | ISYEEDQRQGAEPRKNFVKPNETKTYFWKV |
| | QHHMAPTKDEFDCKAWAYFSDVDLEKDVHS |
| | (SEQ ID NO: 5) |
| 1941-1980 | IRWYLLSMGSNENIHSIHFS |
| | GHVFTVRKKEEYKMALYNLY |
| | (SEQ ID NO: 6) |

C2 DOMAIN

| | |
|---|---|
| 2161-2240 | STLRMELMGCDLNSCSMPLGMESKAISDAQI |
| | TASSYFTNMFATWSPSKARLHLQGRSNAWR |
| | PQVNNPKEWLQVDFQKTMK |
| | (SEQ ID NO: 7) |
| 2281-2330 | VKVFQGNQDSFTPVVNSLDPPLLTRYLRIHP |
| | QSWVHQIALRMEVLGCEAQ |
| | (SEQ ID NO: 8) |

In conclusion, sequence segments of the A3 and C2 domains of fVIII that are recognized by all hemophilia A patients with inhibitors were directly identified. Also, based on the structural similarity between A2 and A3 domains and the relative location of universal epitopes identified here with the sequence regions forming binding sites for inhibitors, candidate regions of the A2 domain that are likely to form universal CD4+ epitopes were identified. A pool of those sequences (synthetic or biosynthetic or directly synthesized by the patient as a result of gene transfer) is useful to induce tolerance to fVIII in hemophilia A patients and in patients with acquired hemophilia.

### REFERENCES

Abbas, A., Murphy, K. and Sher, A, Nature 383:787-793, 1996.

Abbas, A. K., Lichtman, A. and Pober, J. Cellular and Molecular Immunology, 3rd edition, W. B. Saunders Co., 1997.

Abdallah et al., Biol. Cell, 85:1, 1995.

Adelman et al., DNA, 2:183, 1983.

Aledort, L. M., Am. J. Hem. 47:208-217,1994.

Aledort, L. M., Vox Sang 70:77-78, 1996.

Algiman, M., Dietrich, G., Nydegger, U. E., Boieldien, D., Sultan, Y. and Kazatchkine, M. D, Proc. Natl. Acad. Sci. USA 89:3795-3799, 1992.

Allain, J. P., Gaillandre, A. and Lee, H., J. Lab. Clin. Med. 197:791-800, 1981.

Al-Sabbagh, A., P. Nelson, Y. Akselband, R. Sobel, and H. Weiner, Cell Immunol. 171:111-119,1996.

Batlle, J., Gomez, E., Rendal, E., Torea, J., Loures, E., Cousela, M., Vila, P., Sedano, C., Tusell, X., Magallon, M., Quintana, M., Gonzalez-Boullosa, R. and Lopes-Femandez, M. F., Ann. Hematol., 72:321-326, 1996.

Bavaay and Merrifield, “The Peptides,” eds. E. Gross and F. Meienhofer, Vol. 2 (Academic Press, 1980) pp. 3-285.

Bellone M, Karachunski P, Ostlie N, Lei S and Conti-Tronconi B M., Eur. J. Immunol. 24:799-804, 1994.

Bellone M, Karachunski P, Ostlie N, Lei S and Conti-Tronconi B M., Scand. J. Immunol. 41:135-140, 1995.

Benjamini et al. (eds.), Immunology:A Short Course, John Wiley & Sons, Inc., 3rd ed. (1996).

Bi, L., Lawler, A. M., Antonarakia, S., High, K., Gearhart, J. and Kazazian, H. Jr., Nature Genet., 10:119-123, 1995.

Bi, L., Sarkar, R., Naas, T., Lawler, A. M., Pain, J., Shumaker, S. L., Bedian, V. and Kazazian, H. H. Jr., Blood 88:3446-3450, 1996.

Bouvry, P. and Recloux, P., Haematologica 79:550-556, 1994.

Bray, G. L., Kroner, B. L., Arkin, S., Aledort, L. W., Hilgartner, M. W., Etster, M. E., Ragni, M. V. and Goedert, J. J., Am. J. Hematol. 42:375-379, 1993.

Bright, J. J. and Sriram, S., J. Immunol. 161:1772-1777, 1998.

Briner, T. J., Kuo, M. C., Keating, K. M., Rogers, B. L. and Greenstein, J. L., Proc. Nat. Acad. Sci. 90:7608, 1993.

Burdin, N., Van Kooten, C., Galibert, L., Abrams, J. S., Wijdenes, J., Banchereau, J. and Rousset, F., J. Immunol. 154:2533-2544, 1995.

Bums J, Rosenzweig A, Zweiman B, Lisak R P., Cell Immunol. 81:435-440,1983.

Burstein, H. J., Shea, C. M. and Abbas, A. K., J. Immunol. 148:3687-3691, 1992.

Chen, Y., Kuchroo, B. K., Inobe, J., Hafler, D. A. and Weiner, H. L., Science 265:1237-1240, 1994.

Chen, Y., Inobe, J., Marks, R., Gonnella, P., Kuchroo, V. and Weiner, H., Nature 376:177-180,1995.

Chen, Y., Inobe, J.-I., Kuchroo, V. K., Baron, J. L., Janeway, C. A. Jr. and Weiner, H. L., Proc. Natl. Acad. Sci. USA 93:388-391, 1996.

Chicz, R. M., Urban, R. G., Gorga, J. C., Vignali, D. A. A., Lane, W. S. and Strominger, J. L., J. Exp. Med., 178:27-47, 1993.

Choe, S., Bennett, M. J., Fujii, G., Cunni, P. M. G., Kantardjieff, K. A., Collier, R. J. and Eisenberg, D., Nature 357:216, 1992.

Constant, S. L. and Bottomly, K., Ann. Rev. Immunol. 15:297-322, 1997.

Conti-Fine, B. M., McLane, K. E. and Lei, S., Ann. Rev. Biophys. Biomol. Struct. 25:197-229, 1996.

Conti-Fine, B. M., M. P. Protti, M. Bellone, and J. F. Howard, Jr., 1997. Myasthenia Gravis: The Immunobiology of an Autoimmune Disease. R. G. Landes Company, Austin. 230 pp., 1997.

Crea et al., Proc. Natl. Acad. Sci. U.S.A., 75:5765, 1978.

Cresswell, P., Ann. Rev. Immunol. 12:259-294, 1994.

Critchfield, J. M., Racke, M. K., Zuniga-Pflucker, J. C., Cannella, B., Rainse, C. S., Goverman, J. and Lenardo, M., Science 263:1139-1143, 1994.

de Waal Malefyt, R., Yssel, H. and de Vries, J. E., J. Immunol. 150:4754-4765, 1993.

de Wit, D., Van Mechelen, M., Reylandt, M., Figuerido, A. C., Abramovitz, D., Goldman, M., Bazin, H., Urbain, J. and Leo, O., J. Exp. Med. 175:9-14, 1993.

Dick, A., Y. Cheng, A. McKinnon, J. Liversidge, and J. Forrester, Brit. J. Ophthalmol. 77:171-175, 1993.

Dietheim, B., Raju, R., Okita, D. K. and Conti-Fine, B. M., J. Infect. Dis. 175:382-391,1997.

Diethelm-Okita, B., Okita, D. K., Banaszak, L. and Conti-Fine, B. M., J. Infect. Dis. 181:1001-9,2000.

Ding, L. and Shevach, E. M., J. Immunol. 148:3133-3139, 1992.

Ding, L., Linsley, P. S., Huang, L. Y., Germain, R. N. and Shevach, E. M., J. Immunol. 15:1224-1234, 1993.

Donnelly et al., Ann. N.Y. Acad. Sci., 772:40, 1995.

Ehrenforth, S., Kreuz, W., Schaffer, I., Linde, R., Funk, M., Gungor, T., Krackhardt, B. and Komhuber, B., Lancet 339:594-598, 1992.

Enk, A. H., Angeloni, V. L., Udey, M. C. and Katz, S. I., J. Immunol. 151:2390-2398, 1993.

Friedman, A. and Weiner, H. L., Proc. Natl. Acad. Sci. USA 91:6688-6692, 1994.

Genain, C. P., Abel, K., BeLmar, N., Villinger, F., Rosenberg, D. P., Linington, C., Raine, C. S. and Hauser., S. L., Science 274:2054-2057, 1996.

Gilles, J. G. G., Arnout, J., Vermylen, J. and Saint-Remy, J-M. R., Blood 8:2452-2461,1993.

Gilles, J. G. and Saint-Remy, J-.R., J. Clin. Invest., 94:1496-1505, 1994.

Goeddel et al., Nucleic Acids Res., 8:4057 (1980).

Gorham, J. D., Guler, M. L., Fenoglio, D., Gubler, U. and Murphy, K. M., J. Immunol. 161:1664-1670,1998.

Gregerson, D., Obritsch, W. and Donoso, L., J. Immunol. 151:5751-5761, 1993.

Groux, H., Bigler, M., de Vries, J. E. and Roncarolo, M.-G., J. Exp. Med. 184:19-29,1996.

Healey, J. F., Lubin, I. M., Nakai, H., Sarnko, E. L., Hoyer, L. W., Scandella, D. and Lollar, P., J. Biol. Chem., 270(24):14505-14509, 1995.

Heinrickson, S. and Meredith, B., Anal. Biochem. 136:65-74, 1983.

Ho, P. C., Mutch, D. A., Winkel, K. D., Saul, A. J., Jones, G. L., Doran, T. J. and Rzepczyk, C. M., Eur. J. Immunol. 20:477-483, 1990.

Hoffman R W, Takeda Y, Sharp G C, Lee D R, Hill D L, Kaneoka H, Caldwell C W., J Immunol. 151:6460-6469, 1993.

Holt, P. and McMenamin, C., Clin. Exp. Allergy 19:255-262, 1989.

Houghten, R. A., Proc. Natl. Acad. Sci. USA 82:5131-5135, 1985.

Hoyer, L. W., Gawryl, M. S. and de la Fuente, B., Immunochemical characterization of factor VIII inhibitors. In: Factor VIII Inhibitors 73-85, 1984.

Hoyer, L. W., N. Engl. J. Med. 330:38-47, 1994.

Hoyer, L. W. The incidence of factor VIII inhibitors in patients with severe hemophilia A. In: Inhibitors to Coagulation Factors. Aledort, L. M., Hoyer, L. W., Lusher, J. M., Reisner, H. M., and White, G. C., eds., Adv. Exp. Med. Biol. 386:35-45, 1995.

Hunt, D. F., Michel, H., Dickison, T. A., et al., Science 256:1817-1820, 1992.

Husby, S., Mestecky, J., Moldoveanu, Z., Holland, S. and Elson., C., J. Immunol. 152:4663-4670, 1994.

Ito Y, Nieda M, Uchigata Y, Nishimura M, Tokunaga K, Kuwata S, Obata F, Tadokoro K, Hirata Y, Omori Y, Juji T., J. Immunol 151:5770-5776, 1993.

Kappler, J., Roehm, N. and Marrack, P., Cell 49:273-280, 1987.

Karachunski, P., Ostlie, N., Okita, D. and Conti-Fine, B. M., J. Clin. Invest. 100:3027-3035, 1997.

Karachunski, P. I., Ostlie, N. S., Okita, D. K., Garman, R. and Conti-Fine, B. M., J. Neuroimmunol. 93:108-121, 1999.

Kasper, C. K., Aledort, L. A., Counts, R. B. et al., Thromb. Diath. Haemorrh. 34:869-872, 1975.

Kazazian, H., Jr., Tuddenham, E. and Antonarakis, S., Hemophilia A and parahaemophilia: deficiencies of factors Vm and V. in Medical and Metabolic Basis of Inherited Disease, 7th edn (eds Scriver, C. et al.) 3241-3267 (McGraw-Hill, New York), 1995.

Kindler, V. and Zubler, R. H., J. Immunol. 159:2085-2090, 1997.

King, C., Davies, J., Mueller, R., Lee, M. S., Krahl, T., Yeung, B., O'Connor, E. and Sarvetnick, N., Immunity. 8:601-613, 1998.

Kreuz, W., Escuriola-Ettinghausen, C., Martinez-Sauguer, I., Gungor, T. and Kornhuber, B., Vox Sang 70:2-8, 1996.

Kuper, C., P. Koornstra, D. Hameleers, J. Biewenga, B. J. Spit, A. M. Duijvestijn, P. J. van Breda Vriesman, and T. Sminia, Immunol. Today 13:219-224, 1992.

Kuwana M, Medsger T A Jr, Wright T M., J. Clin. Invest. 96:586-596, 1995.

Lacy, D. B., Tepp, W., Cohen, A. C., DasGupta, B. R. and Stevens, R. C., Nature Struct. Biol. 5:898-902, 1998.

Lawn et al., Nucleic Acids Res., 9:6103, 1981.

Lee, C., Recombinant clotting factors in the treatment of hemophilia. Thrombosis and Hemostasis. F. K. Schattauer Verlagsgesellschaft mbH (Stuttgart) 82:516-524, 1999.

Letterio, J. L. and A. B. Roberts, Ann. Rev. Immunol. 16:137-161, 1998.

Liblau, R. S., Singer, S. M. and Mc Devitt, H. O., Immunol. Today 1:34-38, 1995.

Liu, L., and G. G. MacPherson., J. Exp. Med. 177:1299-1307, 1993.

Lollar, P., Adv. Exp. Med. Biol. 386:3-17, 1995.

Lollar, P., Thromb. Haemost. 82:505-508, 1999.

Lusher, J. M., Reisner, H. M. and White, G. C., eds. Adv. Exp. Med. Biol. 386:35-45, 1995.

Ma, C. G., Zhang, G.-X., Xiao, B-G., Link, J., Olsson, T. and Link, H., J Neuroimmunol. 58:51-60, 1995.

Macatonia, S. E., Doherty, T. M., Knight, S. C. and O'Garra, A., J. Immunol, 150:3755-3765, 1993.

Madden, D. R., Ann. Rev. Immunol. 13:587-622, 1995.

Malisan, F., Briere, F., Bridon, J. M., Harindranath, N., Mills, F. C., Max, E. E., Banchereau, J. and Martinezvaldez, H., J. Exp. Med. 183:937-947, 1996.

Manfredi, A. A., Protti, M. P., Wu, X. D., Howard, J. F. Jr. and Conti-Tronconi, B. M., Neurology 42:1092-1100, 1992.

Manfredi A A, Protti M P, Dalton M W M, Howard Jr, J F, Conti-Tronconi B M., J. Clin. Invest. 92:1055-1067, 1993.

Manfredi, A. M. H. Yuen, L. Moiola, M. P. Protti and B. M. Conti-Tronconi, J. Immunol. 152:4165-4174, 1993.

Mariani, G., Hilgartner, M., Thompson, A. R., Tusell, J., Manlo-Johnson, M., Ghierardini, A., Bellocco, R. and Brackrnann, H. H., Adv. Exp. Med. Biol. 386:201, 1995.

Matzinger, P., Ann. Rev. Immunol. 12:991-1045, 1994.

McMillan, C. W., Shapiro, S. S., Whitehurst, D., Hoyer, L. W., Rao, A. V. and Lazerson, J., Blood 71:344, 1988.

McMullen B A, Fujikawa K, Davie E W, Hedner U, Ezban M., Protein Sci. 4:740-6, 1995.

Meienhofer in “Hormonal Proteins and Peptides,” ed.; C. H. Li, Vol. 2 (Academic Press, 1973), pp. 48-267.

Merrifield, J. Am. Chem. Soc., 85:2149, 1963.

Metzler, G. and Wraith, D. C., Int. Immunol. 5:1159-1169, 1993.

Miller, A., Lider, O., Abramsky, O. and Weiner, H. L., Eur. J. Immunol. 24:1026-32, 1994.

Miller, S. D. and Karpus, W. J., Immunol. Today 8:356-361, 1994.

Moiola L, Karachunski P, Protti M, Howard J, Conti-Tronconi B M., J. Clin. Invest. 93:1020-1028, 1994.

Molling, J. Mol. Med., 75:242, 1997.

Morel-Kopp M C, Tchernia G, Lambert T, Verdy E, Schlegel N, Dreyfus M, Chateauvieux L, Kaplan C., Blood 80:49a, 1992.

Moudgil, K. D., Sercarz, E. E. and Grewal, I. S., Immunol. Today 19:217-220, 1998.

Mowat, A., Immunol. Today 8:93-98, 1987.

Muchitsch, F.-M., Turecek, P. L., Gritsch, H., Richter, G., Pichler, T. and Schwarz, H. P., Thromb. Haemost. 77:230, 1997.

Mullis et al., Cold Spring Harbor Symp. Quant. Biol., 51, 263 (1987); Erlich, ed., PCR Technology, (Stockton Press, NY, 1989).

Naylor, J., Banke, A., Hassock, S., Green and Glennelli, G., Hum. Molec. Genet. 2:1773-1779, 1993.

Neutra, M., E. Pringult, and J. Kraehenbuhl, Ann. Rev. Immunol. 14:275-300, 1996.

Newman, S. P. in Aerosols and the Lung, Clarke, S. W. and Davia, D. eds., pp. 197-224, Butterworths, London, England, 1984.

Nilsson, I. M., Bemtorp, E., Zettervall, O. and Dahlback, B., Blood 75:378-83, 1990.

Nilsson, I. M., Bemtorp, E. and Zettervall, O. New Engl. J. Med. 318:947-950, 1998.

Norman, P. S., Ohman, J. L. Jr., Long, A. A., Gefter, P. S., Shaked, A., Wood, R. A., Eggleston, P. A., Hafner, K. B., Rao, P., Lichtenstein, L. M., Jones, N. H. and Nicodemus, C. F., Amer. J Res. Crit. Med. 154:1623, 1996.

Nossal, G., Ann. Rev. Immunol. 13:1-27, 1995.

O'Garra, A., Steinman, L. and Gijbels, K., Curr. Opin. Immunol. 9:872-883, 1997.

O'Garra A., Immunity. 8:275-283, 1998.

Panina-Bordignon, P., Tan, A., Termijtelen, A., Demotz, S. Corradin, G. arid Lanzavecchia, A., Eur. J. Immunol. 19:2237-2242, 1989.

Pardoll et al., Immunity 3:165, 1995.

Paul, Fundamental Immunology, 3rd ed., Raven Press (1993).

Pemberton, S., Lindley, P., Ziatsev, V., Card, G. Tuddenham, E. G. D. and Kemball-Cook, G., Blood 89:2413-2421, 1997.

Pratt K P, Shen B W, Takeshima K, Davie E W, Fujikawa K, Stoddard B L., Nature 402:439442, 1999.

Protti, M. P., Manfredi, A. A., Straub, C., Howard, J. F. Jr. and Conti-Tronconi, B. M., Proc. Natl. Acad. Sci. USA 37:7792-7796, 1990.

Protti, M. P., Manfredi, A. A., Horton, B. M., Bellone, M. and Conti-Tronconi, B. M., Immunol. Today 14:363-368, 1993.

Qian, A., Kazazian, H. Jr. and Hoyer, L., Blood, 88:656a, 1996.

Qian, A., Scott, D. and Hoyer, L., Blood, 90:599a, 1997.

Qian, J., Kazazian, H. Jr. and Hoyer, L., Blood 88:656a, 1996.

Qian, J., Scott, D. and Hoyer, L., Blood 90:599a, 1997.

Qian J, Collins M, Hoyer L W., Blood 92:709a, 1998.

Qian, J., Borovok, M., Lei, B., Kazazian, H. H. and Hoyer, L. W., Thromb. Haemost. 81:240-244, 1999.

Raju, R., Navaneethan, D., Okita, D. K., Diethelm-Okita, B., McCormick, D. and Conti-Fine, B. M., Eur. J. Iminunol. 25:3207-3214, 1995.

Raju, R., B. Diethelem-Okita, B., D. K. Okita, and B. M. Conti-Fine, J. Autoimmunity 9:79-88, 1996.

Reding, M. T., Wu, H., Krampf, M., Okita, D. K., Diethelm-Okita, B. M., Key, N. S. and Conti-Fine, B. M., Thromb. Haemost. 82:509-515, 1999.

Reding M T, Wu H, Krampf M, Okita D K, Diethelm-Okita B M, Christie B A, Key N S, Conti-Fine B M., Thromb. Haemost. 84:643-52, 2000.

Reece, J. C., Geysen, H. M. and Rodda, S. J., J. Immunol. 151:6175-6184, 1993.

Reisner, H. M., Clar, A. and Levin, L., Immunogenetics of the human immune response to factor VIII. Inhibitors to Coagulation Factors, Aledort, L. M., ed., Plenum Press, NY, 65, 1995.

Romagnani, S., Immunol. Today 18:263-266, 1997.

Rousset, F., Peyrol, S., Garcia, E., Vezzio, N., Andujar, M., Grimaud, J. A. and Banchereau, J., Int. Immunol. 7:1243-1253, 1995.

Rudensky, A. Y., Preston-Hurburt, P., Hong, S. C., Barlow, A. and Janeway, C. A. Jr., Nature, 353:622-627, 1991.

Sadler, J. and Davie, E. W., Hemophilia A, hemophilia B and vonWillebrand's disease. In The Molecular Basis of Blood Disease. (eds. Stamatoyanopolous, G. et al.) 575-630 (W. B. Saunders, Philadeplphia), 1987.

Sambrook et al., Molecular Cloning: A Laboratory Manual, Cold Spring Harbor, N.Y. (1989).

Scandella, D., Kessler, C., Esmon, P., Hurt, D., Courter, S., Gomperts, E., Felch, M. and Prescott, R., Adv. Exp. Med. Biol., 386:47-63, 1995.

Scanella, D., Vox Sang, 70(suppl. 1):9-14, 1996.

Schwartz. R. Acquisition of Immunological self-tolerance. Cell 57:1073-1081, 1989.

Seder, R. A. and W. E. Paul, Ann. Rev. Immunol. 12:635-673, 1994.

Seder, R. A., Mart, T., Sieve, M. C., Strober, W., Letterio, J. J., Roberts, A. B. and Kelsall, B., J. Immnunol. 160:5719-5728, 1998.

Shi, H. N., Grusby, M. J. and Nagler-Anderson, C., J. Immunol. 162:5143-5148, 1999.

Shima, M., Nakai, H., Scandella, D. et al., Brit. J Haem., 91:714-721, 1995.

Simmons et al., Science 276:276, 1997.

Stern, L. J., Brown, J. H., Jardetzky, T. S., et al., Nature 368:215-221, 1994.

Stevenson et al., Immunol. Rev., 145:211, 1995.

Stewart et al., Solid Phase Peptide Synthesis W. H. Freeman Co., San Francisco (1969).

Taga, K., Mostowski, H. and Tosato, G., Blood 81:2964-2971, 1993.

Trentham, D. E., Dynesius-Trentham, R. A., Orav, E. J., Combitchi, D., Lorenzo, C., Sewell, K. L., Hafler, D. A. and Weiner, H. L., Science 261:1727-1730, 1993.

Triemstra, M., Rosendaal, F. R., Smit, C., Van der Ploeg, H. M. and Briet, E., Annals Int. Med. 123:823-827, 1995.

Umland, T. C., Wingert, L. M., Swaminathan, S., Furey, W. F., Schmidt, J. J. and Sax, M., Nature Struct. Biol. 4:788-792, 1997.

Vardhachary, A. S., Perdow, S. N., Hu, C., Ramanarayanan, M. and Salgame, P., Proc. Natl. Acad. Sci. USA 94:5778-5783, 1997.

Vehar, G. A., Keyt, D., Eaton, D., Rodriguez, H., O'Brien, D. P., Ratblat, F., Opperman, H., Keck, R., Wood, W. I. and Harkins, R. N., Nature 312:337-342, 1984.

Viera et al., Meth. Enzymol. 153:3 (1987).

von Herrath, M. G., Dyrberg T. and Oldstone, M. B. A., J. Clin. Invest. 98:1324-1331, 1996.

Wang, Z. Y., Qiao, J. and Link, H., J. Neuroimmunol. 44:209-214, 1993.

Wang, Z-Y., Okita, D., Howard, J. and Conti-Fine, B. M., Neurology 48:1643-1653, 1997.

Wang Z Y, Okita D K, Howard J F, Conti-Fine B M., Neurology 50:1045-1054, 1998.

Watts, C., Ann. Rev. Immunol. 15:821-850, 1997.

Weigle, W. O. and Romball, C. G., Immunol. Today 18:533-538, 1997.

Weiner, H. L., Mackin, G. A., Matsui, M., Orav, E. J., Khoury, S. J., Dawson, D. M. and Hafler, D. A., Science 259:1321, 1993.

Weiner, H., Friedman, A., Miller, A., Khoury, S. J., Al-Sabbagh, A., Santos, L., Sayegh, M., Nussenblatt, R. B., Trenthan, D. E. and Hafler, A. D., Ann. Rev. Immunol. 12:809-837, 1994.

Weiner, H. L., Immunol. Today 18:335-343, 1997.

Wu, B., Deng, C., Goluszko, E. and Christadoss, P., J. Immunol. 159:3016-3023, 1997.

Yang et al., Mol. Med. Today 2:476, 1996.

Yuen, M. H., K. Macklin and Bianca M. Conti-Fine, J. Autommunity 9:67-77, 1996.

Zaitseva I, Zaitseva V, Card G, Moshkov K, Bax B, Ralph A, Lindley P., J. Biol. Inorg. Chem. 1:15, 1996.

Zhang, X., Brunner, T., Carter, L., Dutton, R. W., Rogers, P., Bradley, L., Sato, T., Reed, J. D., Green, D. and Swain, S. L., J. Exp. Med. 185:1837-1849, 1997.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. An isolated and purified peptide comprising the amino acid sequence KTWVHYIAAEEEDWDYAPLVLAPDDR (SEQ ID NO:1), or an immunogenic fragment or variant thereof.

2. An isolated and purified peptide comprising the amino acid sequence ITDVRPLYSRRLPKGVKHLKDFPILPGEIFKYKWTVTVEDGPTKSD PRCLTRYYSS (SEQ ID NO:2), or an immunogenic fragment or variant thereof.

3. An isolated and purified peptide comprising the amino acid sequence AYWYILSIGAQTDFLSVFFSGYTFKHKMVYEDTLTLF (SEQ ID NO:3), or an immunogenic fragment or variant thereof.

4. An isolated and purified peptide comprising the amino acid sequence VEMKKEDFDIYDEDENQSPRSFQKKTRHYFIAAVERLWDYGMSSS PHVLRNRAQSGSVPQ (SEQ ID NO:4), or an immunogenic fragment or variant thereof.

5. An isolated and purified peptide comprising the amino acid sequence ISYEEDQRQGAEPRKNFVKPNETKTYFWKVQHHMAPTKDEFDCK AWAYFSDVDLEKDVHS (SEQ ID NO:5), or an immunogenic fragment or variant thereof.

6. An isolated and purified peptide comprising the amino acid sequence IRWYLLSMGSNENIHSIHFSGHVFTVRKKEEYKMALYNLY (SEQ ID NO:6), or an immunogenic fragment or variant thereof.

7. An isolated and purified peptide comprising the amino acid sequence STLRMELMGCDLNSCSMPLGMESKAISDAQITASSYFATWS PSKARLHLQGRSNAWRPQVNNPKEWLQVDFQKTMK (SEQ ID NO:7), or an immunogenic fragment or variant thereof.

8. An isolated and purified peptide comprising the amino acid sequence VKVFQGNQDSFTPVVNSLDPPLLTRYLRIBQSWVHQIALRMEVL GCEAQ (SEQ ID NO:8), or an immunogenic fragment or variant thereof.

9. The peptide of any one of claims 1 to 8 which comprises at least one universal immunodominant region CD4+ epitope sequence.

10. The peptide of any one of claims 1 to 8 which has at least 7 and no more than 40 residues.

11. A tolerogen comprising any one of the peptides of claim 1 to 10, or a combination thereof, combined with a physiologically acceptable, non-toxic liquid vehicle, effective to tolerize a mammal to factor VIII, a biologically active fragment thereof, or a functional equivalent thereof.

12. The tolerogen of claim 11 which is adaptable for nasal administration.

13. The tolerogen of claim 11 which is adaptable for administration to the respiratory tract.

14. The tolerogen of claim 11 which is adaptable for intravenous administration.

15. The tolerogen of claim 11 which is adaptable for subcutaneous administration.

16. The tolerogen of claim 11 which is adaptable for oral administration.

17. The tolerogen of claim 11 which is in a sustained release dosage form.

18. A method of preventing or inhibiting aberrant, pathogenic or undesirable antibody production or antibody binding which is specific for factor VIII, a biologically active fragment thereof, or a functional equivalent thereof, in a mammal, comprising: administering to the mammal a dosage form comprising an effective amount of any one of the peptides of claims 1 to 10 or a combination thereof.

19. A method of preventing or inhibiting the priming or activity of T cells specific for factor VIII, a biologically active fragment thereof, or a functional equivalent thereof, of a mammal, comprising: administering to the mammal a dosage form comprising an effective amount of any one of the peptides of claims 1 to 10 or a combination thereof.

20. A method of enhancing the activity or increasing the levels of modulatory T cells that inhibit the immune response to factor VIII, a biologically active fragment thereof, or a functional equivalent thereof, of a mammal, comprising: administering to the mammal a dosage form comprising an effective amount of any one of the peptides of claims 1 to 10 or a combination thereof.

21. A method to tolerize a mammal to factor VIII, a biologically active fragment thereof, or a functional equivalent thereof, comprising: administering to the mammal a dosage form comprising an effective amount of any one of the peptides of claims 1 to 10 or a combination thereof.

22. The method of any one of claims 19 to 21 wherein the administration is effective to prevent or inhibit the synthesis of antibody specific for factor VIII, a biologically active fragment thereof, or a functional equivalent thereof, reduce or inhibit the amount of antibody specific for factor VIII, a biologically active fragment thereof, or a functional equivalent thereof, or the affinity of the antibody for factor VIII, a biologically active fragment thereof, or a functional equivalent thereof.

23. The method of any one of claims 18 to 21 wherein the mammal is a human.

24. The method of any one of claims 18 to 21 wherein the dosage form is administered to the respiratory tract.

25. The method of any one of claims 18 to 21 wherein the dosage form is administered subcutaneously.

26. The method of any one of claims 18 to 21 wherein the dosage form is administered nasally.

27. The method of any one of claims 18 to 21 wherein the dosage form is administered intravenously.

28. The method of any one of claims 18 to 21 wherein the dosage form is administered orally.

29. The method of any one of claims 18 to 21 wherein the dosage form is in sustained release dosage form.

30. A therapeutic method, comprising: administering to a mammal having an indication or disease characterized by a decreased amount or a lack of biologically active factor VIII and which mammal is subjected to exogenous introduction of factor VIII, a biologically active fragment thereof, or a functional equivalent thereof, a dosage form comprising an amount of any one of the peptides of claims 1 to 10 or a combination thereof effective to inhibit or reduce antibody inhibitors of factor VIII.

31. The method of claim 30 wherein the exogenous introduction of factor VIII, a biologically active fragment thereof, or a functional equivalent thereof, is via a recombinant virus which encodes factor VIII, a biologically active fragment thereof, or a functional equivalent thereof.

32. The method of claim 31 wherein the virus is a retrovirus.

33. The method of claim 31 wherein the virus is an adenovirus.

34. The method of claim 30 or 31 wherein the dosage form is administered to the respiratory tract.

35. The method of claim 30 or 31 wherein the dosage form is administered subcutaneously.

36. The method of claim 30 or 31 wherein the dosage form is administered nasally.

37. The method of claim 30 or 31 wherein the dosage form is administered intravenously.

38. The method of claim 30 or 31 wherein the dosage form is administered orally.

39. The method of claim 30 or 31 wherein the dosage form is in sustained release dosage form.

40. The method of any one of claims 18 to 21 or 30 wherein the administration of the peptide does not increase synthesis of pathogenic antibody to factor VIII, a biologically active fragment thereof, or a functional equivalent thereof.

41. The method of claim 30 wherein the mammal is subjected to plasmapheresis.

42. The method of claim 41 further comprising administering an agent that inhibits B cell activation.

43. The method of claim 18 to 21 or 30 wherein the peptide is KTWVHYIAAEEEDWDYAPLVLAPDDR (SEQ ID NO:1), ITDVRPLYSRRLPKGVKHLKDFPILPGEIFKYKWTVTVEDGPTKSD PRCLTRYYSS (SEQ ID NO:2), AYWYILSIGAQTDFLSVFFSGYTFKH KMVYEDTLTLF (SEQ ID NO:3), VEMKKEDFDIYDEDENQSPRSFQKKTRHYFIAAVERLWDYGMSSS PHVLRNRAQSGSVPQ(SEQ ID NO:4), ISYEEDQRQGAEPRKNFVKPNETKTYFWKVQHHMAPTKDEFDCK AWAYFSDVDLEKDVHS (SEQ ID NO:5), YDEDENQSPRSPQKKTRHYFI IRWYLLSMGSNENIHSIFSGHVFTVRKKEEYKMALYNLY (SEQ ID NO:6), STLRMELMGCDLNSCSMPLGMESKAISDAQI TASSYFTNMFATWSPSKARLHLQGRSNAWRPQVNNPKEWLQVDF QKTMK (SEQ ID NO:7), VKVFQGNQDSFTPVVNSLDPPLLTRYLRIHPQSWVHQIALRMEVL GCEAQ (SEQ ID NO:8), or an immunogenic fragment thereof.

44. The peptide of claim 4 which comprises SEQ ID NO: 54, SEQ ID NO: 55, SEQ ID NO: 56, SEQ ID NO: 57, SEQ ID NO: 58, SEQ ID NO: 59 or SEQ ID NO: 60.

45. The peptide of claim 5 which comprises SEQ ID NO: 14, SEQ ID NO: 15, SEQ ID NO: 16, SEQ ID NO: 17, SEQ ID NO: 18, SEQ ID NO: 19 or SEQ ID NO: 20.

46. The peptide of claim 6 which comprises SEQ ID NO: 29, SEQ ID NO: 30, SEQ ID NO: 31, SEQ ID NO: 32, or SEQ ID NO: 33.

47. The peptide of claim 7 which comprises SEQ ID NO:61, SEQ ID NO: 37, SEQ ID NO: 38, SEQ ID NO: 39, SEQ ID NO: 40, SEQ ID NO: 41, SEQ ID NO: 42, or SEQ ID NO: 43.

48. The peptide of claim 8 which comprises SEQ ID NO: 47, SEQ ID NO: 48, SEQ ID NO: 49, SEQ ID NO: 50, SEQ ID NO: 51, or SEQ ID NO: 52.

49. A therapeutic method, comprising: administering to a mammal having an indication or disease characterized by a decreased amount or a lack of biologically active factor VIII and which mammal is subjected to exogenous introduction of factor VIII, a biologically active fragment thereof, or a functional equivalent thereof, a dosage form comprising an amount of a nucleic acid molecule encoding any one of the peptides of claims 1 to 10 or a combination thereof effective to inhibit or reduceantibody inhibitors of factor VIII.

50. A therapeutic method, comprising: administering to a mammal having an indication or disease characterized by a decreased amount or a lack of biologically active factor VIII and which mammal is subjected to exogenous introduction of DNA encoding factor VIII, a biologically active fragment thereof, or a functional equivalent thereof, a dosage form comprising an amount of a nucleic acid molecule encoding any one of the peptides of claims 1 to 10 or a combination thereof effective to inhibit or reduce antibody inhibitors of factor VIII.

51. A method of preventing or inhibiting aberrant, pathogenic or undesirable antibody production or antibody binding which is specific for factor VIII, a biologically active fragment thereof, or a functional equivalent thereof, in a mammal, comprising: administering to the mammal a dosage form comprising an effective amount of a nucleic acid molecule encoding any one of the peptides of claims 1 to 10 or a combination thereof.

52. A method of preventing or inhibiting the priming or activity of T cells specific for factor VIII, a biologically active fragment thereof, or a functional equivalent thereof, of a mammal, comprising: administering to the mammal a dosage form comprising an effective amount of a nucleic acid molecule encoding any one of the peptides of claims 1 to 10 or a combination thereof.

53. A method of enhancing the activity or increasing the levels modulatory T cells that inhibit the immune response to factor VIII, preventing, a biologically active fragment thereof, or a functional equivalent thereof, of a mammal, comprising: administering to the mammal a dosage form comprising an effective amount of a nucleic acid molecule encoding any one of the peptides of claims 1 to 10 or a combination thereof.

54. A method to tolerize a mammal to factor VIII, a biologically active fragment thereof, or a functional equivalent thereof, comprising: administering to the mammal a dosage form comprising an effective amount of of a nucleic acid molecule encoding any one of the peptides of claims 1 to 10 or a combination thereof.

55. The method of any one of claims 49 to 54 wherein the dosage form comprises a recombinant virus which encodes the peptide.

56. The method of any one of claims 49 to 54 wherein the dosage form comprises DNA.

57. The tolerogen of claim 11 wherein the mammal is a human.

58. The method of any one of claims 49 to 54 wherein the mammal is a human.
